(19) United States
(12) Patent Application Publication
(10) Pub. No.: US 2010/0048705 A1
(43) Pub. Date: Feb. 25, 2010

(54) METHODS OF TREATING AND PREVENTING NEOVASCULARIZATION WITH OMEGA-3 POLYUNSATURATED FATTY ACIDS

(75) Inventors: Lois E. Smith, West Newton, MA (US); Kip M. Connor, Brighton, MA (US); Charles Serhan, Needham, MA (US)

Correspondence Address: DAVID S. RESNICK, NIXON PEABODY LLP, 100 SUMMER STREET, BOSTON, MA, 02110-2131, US

(73) Assignees: CHILDREN'S MEDICAL CENTER CORPORATION, Boston, MA (US); THE BRIGHAM AND WOMEN'S HOSPITAL, INC., Boston, MA (US)

(21) Appl. No.: 12/447,768

(22) PCT Filed: Nov. 9, 2007
(86) PCT No.: PCT/US07/23650

§ 371 Date: Apr. 29, 2009

Related U.S. Application Data
(60) Provisional application No. 60/857,998, filed on Nov. 9, 2006.

(51) Int. Cl. A61K 31/202; A61P 27/02

(52) U.S. Cl. 514/560

(57) ABSTRACT

Disclosed are methods for treating or preventing ocular neovascularization in a subject at risk. The method comprises administering to the subject an effective amount of omega-3 polyunsaturated fatty acid to thereby treat or prevent the ocular neovascularization. This method is suitable for treating or preventing retinopathy of prematurity, retina vein occlusion, sickle cell retinopathy, choroidal neovascularization, radiation retinopathy, microangiopathy, retinal hyperoxia, diabetic retinopathy, and age related macular degeneration. Preferably the methods are applied to premature infants, especially those exposed to high levels of oxygen, to treat or prevent ocular neovascularization results from retinopathy of prematurity. Preferably, the omega-3 polyunsaturated fatty acid is administered at high dose, periodically (e.g. from birth) over a prolonged period of time, until the eye is fully vascularized, or to the age of 1 year. Appropriate routes of administration include oral and intravenous administration. Suitable omega-3 polyunsaturated fatty acids include docosahexaenoic acid and eicosapentaenoic acid. These agents can be administered in a pharmaceutically acceptable carrier, e.g. one which contains an anti-oxidant for the omega-3 polyunsaturated fatty acid.

### CROSS REFERENCE TO RELATED APPLICATIONS

This application claims benefit under 35 U.S.C. §119(e) of U.S. Provisional Application Ser. No. 60/857,998, filed Nov. 9, 2006, the contents of which are incorporated herein in their entirety.

### GOVERNMENT SUPPORT

This invention was made with Government support under grants EY008670, EY017017, EY14811; 5 T32 EY07145 (KMC) P50-DE016191, and GM38765, awarded by the National Institute of Health. The Government has certain rights in the invention.

### FIELD OF THE INVENTION

The invention described herein relates to methods of treatment of various forms of retinopathy in a subject associated with neovascularization by administration of omega-3 polyunsaturated fatty acids.

### BACKGROUND OF THE INVENTION

Ocular neovascularization is the most common cause of blindness in all age groups, being associated with retinopathy of prematurity in children, diabetic retinopathy in working age-adults and age-related macular degeneration in the elderly. Retinopathy of prematurity (ROP) is a potentially blinding eye disorder that primarily affects premature and underweight infants. The smaller a baby is at birth, the more likely that baby is to develop ROP. This disorder usually develops in both eyes, and is one of the most common causes of visual loss in childhood and can lead to lifelong vision impairment and blindness. About 1,100-1,500 infants annually develop ROP that is severe enough to require medical treatment. About 400-600 infants each year in the US become legally blind from ROP.

Diabetic retinopathy is the most common diabetic eye disease and a leading cause of blindness in American adults. It is caused by changes in the blood vessels of the retina. In some cases of diabetic retinopathy, fragile, abnormal blood vessels develop and leak blood into the center of the eye, blurring vision. In others, abnormal new blood vessels grow on the surface of the retina.

Age-related macular degeneration is a degenerative condition of the macula (the central retina). It is the most common cause of vision loss in the United States in those 50 or older, and its prevalence increases with age of an individual. Age-related macular degeneration is caused by hardening of the arteries that nourish the retina. This deprives the sensitive retinal tissue of oxygen and nutrients that it needs to function and thrive. As a result, the central vision deteriorates. Ten percent of age related macular degeneration is caused by neovascularization, where new blood vessels form to improve the blood supply to oxygen-deprived retinal tissue.

These forms of retinopathy are all conditions related to pathological angiogenesis in the eye. The role of protein growth factors in the regulation of angiogenesis is well known, but the role of lipids in this process, while beginning to be elucidated^{2, 3}, is still largely undefined. Docosahexaenoic acid (DHA; C22:6omega-3) and arachidonic acid (AA; C20:4omega-6) are the major polyunsaturated fatty acids found in the retina⁴. DHA and AA are mainly found in neural and vascular cell membrane phospholipids and eicosapentaenoic acid (EPA; C20:5omega-3), the precursor to DHA, is found in retinal vascular endothelium⁵. Polyunsaturated fatty acids are released as free fatty acids by phospholipase A₂, which is induced by ischemia, inflammation, neuroactive compounds, redox balance, and light exposure. Dietary sources of EPA, DHA, and AA contribute substantially with lipids from tissue to a substrate pool for enzymes that convert free polyunsaturated fatty acids to vaso- and immuno-regulatory lipid mediators⁶ which include separate families of bioactive mediators such as eicosanoids from AA, neuroprotectins such as neuroprotectin D1 from DHA, D series resolvins from DHA, and E series resolvins from EPA^{7, 8}.

ROP occurs when abnormal blood vessels grow and spread throughout the retina, the tissue that lines the back of the eye. These abnormal blood vessels are fragile and often leak, scarring the retina and pulling it out of position. This causes a retinal detachment, which is the main cause of visual impairment and blindness in ROP. Several complex factors are thought responsible for the development of ROP. Development of the eye begins at about 16 weeks of pregnancy, when the blood vessels of the retina begin to form at the optic nerve in the back of the eye. The blood vessels grow gradually toward the edges of the developing retina, supplying oxygen and nutrients. The eye develops rapidly during the last 12 weeks of a pregnancy. The retinal blood vessel growth is mostly complete when a baby is born full-term as the retina usually finishes growing a few weeks to a month after birth. However, premature birth that occurs before these blood vessels have reached the edges of the retina, can halt the normal vessel growth. As such, the edges of the retina—the periphery—may not get enough oxygen and nutrients. It is thought that the periphery of the retina then sends out signals to other areas of the retina for nourishment, causing growth of new abnormal vessels. Bleeding from these fragile new blood vessels leads to retinal scarring. Scar shrinkage then pulls on the retina, causing it to detach from the back of the eye.

Retinopathy is modeled in the mouse eye with oxygen-induced vessel loss which precipitates hypoxia-induced retinopathy¹. One-week-old C57BL/6J mice are exposed to 75% oxygen for 5 days and then to room air. A fluorescein-dextran perfusion method is used to assess the vascular pattern. The proliferative neovascular response is quantified by counting the nuclei of new vessels extending from the retina into the vitreous in 6 microns sagittal cross-sections. Cross-sections are also stained for glial fibrillary acidic protein (GFAP). Fluorescein-dextran angiography delineates the entire vascular pattern, including neovascular tufts in flat-mounted retinas. Hyperoxia-induced neovascularization occurs at the junction between the vascularized and avascular retina in the mid-periphery. Retinal neovascularization occurs in all the pups between postnatal day 17 and postnatal day 21. This serves as a reproducible and quantifiable mouse model of oxygen-induced retinal neovascularization for the study of pathogenesis of retinal neovascularization as well as for the study of medical intervention for ROP and other retinal angiopathies in humans. This model allows assessment of retinal vessel loss, vessel re-growth after injury and pathological angiogenesis¹.

### SUMMARY OF THE INVENTION

The present invention provides a method for treating or preventing ocular neovascularization in a subject at risk. The method comprises administering to the subject an effective amount of omega-3 polyunsaturated fatty acid to thereby treat or prevent the ocular neovascularization. Ocular neovascularization associated with retinopathy of prematurity, retina vein occlusion, sickle cell retinopathy, choroidal neovascularization, radiation retinopathy, microangiopathy, retinal hyperoxia, diabetic retinopathy, ablation induced neovascularisation (e.g. ocular) and age related macular degeneration are suitable for such therapy. The present invention also provides a method for preventing irreversible vision loss arising from ocular neovascularization in a subject at risk comprising administering to the subject an effective amount of omega-3 polyunsaturated fatty acid to thereby prevent the ocular neovascularisation from progressing to irreversible vision loss.

One group of subjects suitable for such therapy include premature infants, especially those exposed to high levels of oxygen, which are at increased risk for neovascularization which results from retinopathy of prematurity. In one embodiment, administration is wherein the omega-3 polyunsaturated fatty acid is administered in a regimen over a period of time between birth and the age of one year. In another embodiment administration is periodic, until such a time as the eye is fully vascularized. Other groups of subjects suitable for such therapy include subjects diagnosed with diabetes and subjects over the age of 55.

In one embodiment, administration is over a prolonged period of time (e.g. until symptoms are acceptable reduced or eliminated). In one embodiment administration is oral. In another embodiment administration is intravenous. In another embodiment, the omega-3 polyunsaturated fatty acid is in a pharmaceutically acceptable carrier. In another embodiment, the pharmaceutically acceptable carrier comprises an anti-oxidant. In another embodiment, the omega-3 polyunsaturated fatty acid is administered in a high-dose. In another embodiment, the omega-3 polyunsaturated fatty acid is in an emulsion. In one embodiment, the emulsion is free of plant derived omega-6 fatty acids. In one embodiment, the emulsion comprises fish oil.

In one embodiment, the administered omega-3 polyunsaturated fatty acid is docosahexaenoic acid or eicosapentaenoic acid, or any combination thereof.

### BRIEF DESCRIPTION OF THE DRAWINGS

FIG. 1 contains two bar graphs. Retinas from C57Bl/6 mice fed a diet with a physiologic elevation in either omega-3 or omega-6 polyunsaturated fatty acids were isolated, stained, and flat-mounted after induction of retinopathy. At P17 retinal vasculature stained with lectin-FITC showed more extensive vaso-obliteration (†) and neovascularization (††) in the omega-6 versus omega-3 polyunsaturated fatty acid fed mice (omega-6 n=14, and omega-3 n=27). (a) Vaso obliteration (\p≦0.0001) and (b) neovascularization (‡‡p≦0.0001) was reduced ˜2 fold in the omega-3 versus omega-6 polyunsaturated fatty acid fed mice at P17.

FIG. 2 contains two bar graphs (a and b). After induction of retinopathy, retinas from fat-1 homozygotes and wild type control mice were flat-mounted at P17. Retinal vasculature stained with lectin-FITC shows more extensive retinal vaso-obliteration (*) and neovascularization (**) in wild type versus fat-1 retinas. (a) Vaso-obliteration was reduced ˜2 fold (* p≦0.001) and (b) neovascularization reduced ˜2 fold (** p≦0.001) in the fat-1 expressing mice with higher levels of omega-3 polyunsaturated fatty acids and lower levels of omega-6 polyunsaturated fatty acids compared to controls (WT n=20, and Fat-1 n=16).

FIG. 3 contains two bar graphs (a and b). After exposure to 75% oxygen for 24 hours from P7-P8, retinas from either (a) C57Bl/6 mice given a diet high in either omega-3 or omega-6 polyunsaturated fatty acids (omega-6 n=10, and omega-3 n=10) or (b) Fat-1 mice and controls (WT n=14, and Fat-1 n=7) show equally extensive vaso-obliteration in both the (a) omega-6 polyunsaturated fatty acid and omega-3 polyunsaturated fatty acid fed mice as well as in the (b) Fat-1 and wild type controls at P8.

FIG. 4 is a schematic of resolvins and neuroprotectins biosynthesis from omega-3 polyunsaturated fatty acid. The omega-22 hydroxy-PD1 is the inactivation metabolic of NPD1, a biosynthetic marker of this pathway. RvE2 of the E series EPA resolvins is also a biosynthetic marker identified in the retina.

FIG. 5 contains two spectra, a LC MS/MS spectrum of RvE₂ and a spectrum of omega-22-hydroxy-PD1 obtained from retinal extracts of mice given an omega-3 polyunsaturated fatty acid diet.

FIG. 6 is a tabulation of relative levels of RvE₂ and omega-22-hydroxy-PD1 in retinas of mice on a high omega-3 polyunsaturated fatty acid diet (6 retinas).

FIG. 7 is a collection of three bar graphs. Neither resolvins nor neuroprotectins were identified in retinas of omega-6 polyunsaturated fatty acid fed mice. C57Bl/6 mice were injected i.p. daily P6-P17 with 10 ng of RvD1, RvE1, NPD1 or a Saline/EtOH control (RvD1 n=14, RvE1 n=10, NPD1 n=14 and Saline n=14). (a) is a bar graph which compares vessel loss in RvD1, RvE1 or NPD1 treated mice compared to their vehicle control treated counterparts. A 40% decrease in vessel loss (VO) was observed in RvD1, RvE1 or NPD1 treated mice compared to their vehicle control treated counterparts (*p≦0.001). (b) is a bar graphs which compares neovascularization observed in mice injected i.p. with RvD1, RvE1, or NPD1 compared to vehicle-treated mice (†p≦0.03). There was a 30% decrease in neovascularization (Tufts) in mice injected i.p. with RvD1, RvE1, or NPD1 compared to vehicle-treated mice (†p≦0.03). (c) is a bar graph which compares vaso-obliteration (VO) in mice injected from P5-P8 as above. There was no protective action with either RvE1 or NPD1 treatment on oxygen-induced vessel loss at P8 (RvD1 n=7, RvE1 n=9, NPD1 n=7 and Saline n=6).

FIG. 8 is a bar graph that indicates that mean total retinal TNF-α mRNA expression was increased at P8 and P14 approximately 10-fold in omega-6 fed mice compared to their omega-3 polyunsaturated fatty acid fed counterparts (*p≦0.0001, n=4).

FIG. 9 is a bar graph and a photo of a Western blot probed for TNF-α. Retinal levels of TNF-α were analyzed by Western blot analysis in mice on either omega-3 polyunsaturated fatty acid or omega-6 polyunsaturated fatty acid diets. Mice on the omega-3 polyunsaturated fatty acid diet had a significant decrease in TNF-α protein levels (#p≦0.001, n=4).

FIG. 10 contains two bar graphs. a) indicates the percentage of vaso-obliteration (VO) observed in omega-6 fed pups injected intraperitoneally with either TNF-α receptor fusion protein (etanercept) or a saline control. The injections of TNF-α receptor fusion protein resulted in a significant reduction in vaso-obliteration in omega-6 polyunsaturated fatty acid fed mice compared to saline injected controls (†p≦0.001, n=8). (b) indicates the pathologic neovascularization observed in omega-6 fed pups injected intraperitoneally with either TNF-α receptor fusion protein (etanercept) or a saline control. TNF-α receptor fusion protein treated omega-6 polyunsaturated fatty acid fed mice had a significant reduction in pathologic neovascularization compared to saline injected controls (††p≦0.05, n=8).

FIG. 11 is two bar graphs. (a) indicates the percentage vaso-obliteration observed in omega-6 fed pups injected intraocularly with either TNF-α receptor fusion protein (etanercept) or a saline control. Intraocular injections of the TNF-α receptor fusion protein significantly reduce vaso-obliteration compared to fellow saline-injected eye in omega-6 polyunsaturated fatty acid fed mice (p≦0.005, Saline n=10 and anti-TNF-α n=7). (b) indicates the pathologic neovascularization observed in omega-6 fed pups injected intraocularly with either TNF-α receptor fusion protein (etanercept) or a saline control. Intraocular administration of the TNF-α receptor fusion protein also significantly improved neovascularization in these mice (p≦0.05, Saline n=10 and anti-TNF-α n=7).

FIG. 12 is a collection of six bar graphs. (a) is two graphs, the left indicating the percentage vaso-obliteration, and the right indicating the pathologic neovascularization, observed in P17 pups, fed with omega-6 versus omega-3 long chain polyunsaturated fatty acids, with feeding having begun at P0. (b) is two graphs, the left indicating the percentage of vaso-obliteration, and the right indicating the pathologic neovascularization, observed in P17 pups, fed with omega-6 versus omega-3 long chain polyunsaturated fatty acids, with feeding having begun at P12. (c) is two graphs, the left indicating the percentage of vaso-obliteration, and the right indicating the pathologic neovascularization, observed in P17 pups, fed with omega-6 versus omega-3 long chain polyunsaturated fatty acids, with feeding having begun at P15.

### DETAILED DESCRIPTION OF THE INVENTION

Aspects of the present invention stem from the finding that increasing the omega-3 polyunsaturated fatty acid in a subject experiencing conditions which promote ocular neovascularization, protects the subject from development of ocular neovascularization, and can also reverse the effects/pathology of ocular neovascularization after onset. Ocular neovascularization occurs when abnormal blood vessels grow and spread throughout the retina, the tissue that lines the back of the eye. These abnormal blood vessels are fragile and often leak, scarring the retina and pulling it out of position. This causes a retinal detachment, which is the main cause of visual impairment and blindness in people that experience ocular neovascularization such as ROP. Irreversible vision loss occurs when there is progression from retinal/ocular neovascularization to cicatrisation and retinal detachment. Results from experiments detailed in the Examples section below indicate that increasing the omega-3 polyunsaturated fatty acids in a subject can be used to reduce and/or prevent pathological angiogenesis associated with retinopathy, and may also be useful in reducing and preventing pathological other (non-ocular) forms of pathological angiogenesis. The results further indicate that the greater the increase in the omega-3 polyunsaturated fatty acid in the subject, especially in the effected tissue of the subject, the greater the therapeutic effects. As such, the methods described herein find use in the prevention and/or amelioration of retinal injury/pathologies resulting from retinal occlusion followed by neovascularization. The methods described herein can be applied to prevention (either complete or incomplete) of ocular neovascularization in a subject from progressing to irreversible vision loss. In this respect, all methods described herein for treating or preventing ocular neovascularization in a subject are equally applicable to methods for preventing irreversible vision loss (e.g. reduction in vision loss or complete prevention of vision loss) arising from ocular neovascularization in a subject.

One aspect of the present invention relates to methods for treating or preventing ocular neovascularization in a subject at risk by increasing the omega-3 polyunsaturated fatty acids in the subject. A preferred way to increase the subject's omega-3 polyunsaturated fatty acids is through administration of an agent which increases the subject's omega-3 polyunsaturated fatty acid.

A variety of such agents are known to the skilled practitioner, the most widely known being omega-3 polyunsaturated fatty acids or precursor's thereof. Suitable agents, e.g. suitable omega-3 polyunsaturated fatty acids, are readily determined by the skilled practitioner. The resolvins (resolution phase interaction products) and neuroprotectins (including neuroprotectin D1, also known as protectin D1) are omega-3 polyunsaturated fatty acid bioactive products derived from EPA and DHA. Resolvins and neuroprotectins useful in the methods and compositions as disclosed herein, and methods of their synthesis, are disclosed in International Patent Applications WO04/014835 and WO05/105025 and U.S. Patent Applications 2005/0238589, 2006/0293288, 2005/0238589, 2005/0261255 and 2004/0116408 which are incorporated herein by reference in their entirety.

In addition agonists or analogues of omega-3 polyunsaturated fatty acids can be used. Suitable omega-3 polyunsaturated fatty acids include, without limitation eicosapentaenoic acid (EPA), docosahexaenoic acid (DHA), alpha-linolenic acid (ALA), and docosapentaenoic acid (otherwise known as clupanodonic acid, commonly called DPA). A specific omega-3 polyunsaturated fatty acid may be administered singly or in combination with any such other omega-3 polyunsaturated fatty acid. Likewise, any such agent may be administered singly or in combination with any other such agent(s), to generate an effective amount. In one embodiment, the agent is administered in the complete absence of an agent(s) that increases other types of fatty acids (other than omega-3 polyunsaturated, e.g. omega-6 polyunsaturated fatty acids) in the subject. In another embodiment, the agent is administered in the presence of comparatively lower amounts of an agent(s) that increases other types of fatty acids (other than omega-3 polyunsaturated) in the subject. In one such embodiment administration of omega-3 polyunsaturated fatty acids is alone or in vast excess of omega-6 polyunsaturated fatty acids to promote a decrease in the omega-6:omega-3 ratio in the subject.

### Administration

Appropriate administration of the agent will result in delivery of the desired amount of the agent to the target area or tissue. Administration may result in systemic exposure (e.g. oral or intra venous) or may result in local or topical exposure (e.g. from implants, tissue injection, eye drops). Such administration can be readily determined by the skilled practitioner.

Preferably, the agent is administered in a pharmaceutically acceptable carrier. Such carriers are typically used to promote appropriate delivery of the compounds contained therein without, or with reduced, production of undesirable physiological effects. The composition of the carrier will depend upon a variety of factors, such as the specific agent(s) used, the route and method of administration, and the subject. Such compositions are readily determined by the skilled practitioner. Inclusion of an anti-oxidant which prolongs the effective life of the agent (e.g. omega-3 polyunsaturated fatty acid) may provide particular benefits. Appropriate administration is in a manner compatible with the dosage formulation, the particular condition, disease or injury being treated, and the prescribed regimen.

Appropriate regimens of administration will depend upon the condition being treated, the preferred routes of administration, and the subject themselves, and can be determined by the skilled practitioner on a case by case basis. Examples of suitable regimens include, without limitation, several times per day (e.g. with meals) daily and weekly. Suitable regimens will last as long as necessary to achieve the desired or optimal results. This may be over a period of weeks, months, or longer if necessary. For chronic conditions, it may be necessary to have a longstanding regimen for the lifetime of the subject.

### Dosage

An effective amount administered is an amount sufficient to produce the therapeutic results of treatment or prevention as described herein, will be measurable by an observed therapeutic change in the subject. Often this is referred to as a therapeutically effective amount. A therapeutic change is a change in a measured biochemical, physical or sensory characteristic in a direction expected to alleviate the disease, condition or injury being addressed. This can be determined in a number of ways by the skilled practitioner. One example of such a method of determination is by analysis of the composition of the subject's total omega-3 level, or alternatively of the omega-3 composition of a particular tissue (e.g. retina or other neovascularized tissue) for a determination as to whether the desired increase is achieved by the administration. Another way to determine sufficient amounts is through empirical means (e.g. administration of increasing amounts until symptoms decline).

Therapeutic benefit is produced from administration of a wide range of doses of omega-3 polyunsaturated fatty acid to the subject. The exact dose for optimal results can be determined by the skilled practitioner. Increased benefits are often achieved by administration of high doses of omega-3 to the subject. Experiments detailed in the Examples section below indicate that a subject which has a high level of omega-3, especially when coupled with a low omega-6:omega-3 ratio, enjoys higher levels of protection from pathology than a subject with lower amounts of omega-3, and/or higher ratios of omega-6:omega-3. Such increased omega-3 and decreased ratio of omega-6:omega-3 can be achieved by administration of high doses of omega-3 polyunsaturated fatty acids to a subject.

The dosage of omega-3 polyunsaturated fatty acid can be determined by various means, such as from body weight of the subject or from the subject's dietary intake. For example, it is recommended that the subject receive a dose whereby at least 2% of total fatty acid intake is omega-3 polyunsaturated fatty acid. High doses would be considered to be higher than 2% of total fatty acid intake. A wide range of doses which are greater than 2% total fatty acid intake would be beneficial, starting with 0.1% incremental increases (e.g. ≧2.1%, ≧2.2% etc), or 0.5% incremental increases (e.g. ≧2.5%, ≧3%, ≧3.5% etc.). Beneficial results can also result from dosage that is 10% or more of total fatty acid intake. Higher doses, over 10%, would also provide some benefit in certain situations. Such dosage is to be administered by methods of administration and over periods of time as discussed herein.

Examples of doses include without limitation, ≧100 mgs omega-3 polyunsaturated fatty acid/day, ≧150 mgs omega-3/day, ≧180 mgs omega-3/day, ≧200 mgs omega-3/day, ≧250 mgs omega-3/day, ≧300 mgs omega-3/day, ≧350 mgs omega-3/day, ≧400 mgs/day, ≧450 mgs/day, ≧500 mgs omega-3 polyunsaturated fatty acid/day.

In determining dosage, the skilled practitioner will often take the subject's weight into consideration. As such, dosage which deliver a specified amount of omega-3 polyunsaturated fatty acid/kilogram subject weight (mg omega-3/kg subject) are used. In one embodiment, the formulation is such to deliver 20 mg omega-3/kg subject. Other such formulations include, without limitation, ≧25, ≧30, ≧35, ≧40, ≧45, ≧50, ≧55, ≧60, ≧65, ≧70, ≧75, ≧80, ≧85, ≧90, ≧95, ≧100, ≧105, ≧110, ≧115, ≧120, ≧125, ≧130, ≧135, ≧140, ≧145, and ≧150 mg omega-3/kg subject.

It has been established that doses as high as 10 g/day omega-3 polyunsaturated fatty acid in an individual can be tolerated without detriment. Therapeutic benefit may be achieved by doses ranging from ≧2 g/day, ≧2.5 g/day, ≧3 g/day, ≧3.5 g/day, ≧4 g/day, ≧4.5 g/day, ≧5 g/day, ≧5.5 g/day, ≧6 g/day, ≧6.5 g/day, ≧7 g/day, ≧7.5 g/day, ≧8 g/day, ≧8.5 g/day, ≧9 g/day, ≧9.5 g/day, and ≧10 g/day. A high dose in an adult would be one that exceeds 3 g/day. Under certain conditions, doses significantly higher than 10 g/day may be of benefit. All approximate dosage described herein as a daily dosage may be broken up into correspondingly lower doses administered several times per day. Such formulations can be delivered by the various means, and in the various intervals and regimens described herein.

A high dose of omega-3 polyunsaturated fatty acid may be in the form of concentrated oil such as that disclosed in WO 2005/046669, the contents of which are herein incorporated by reference. If using a concentrated oil, it is preferred that other components (e.g., non-omega-3 fatty acids and contaminants) be removed, as some contents may be detrimental to the subject or inhibitory to the therapy. A highly concentrated amount of omega-3 polyunsaturated fatty acid may be in the form of an emulsion. Alternatively, non-emulsion types of formulations may be used.

The omega-3-fatty acids may be from marine or synthetic origin. For example, a suitable source of omega-3 fatty acids is fish or seal oil. Suitable fish oil sources include cod, menhaden, herring, mackerel, caplin, tilapia, tuna, sardine, pacific saury, krill, salmon, and the like.

It is known that fish oils contain eicosapentaenoic and docosahexanoic acid in the triglyceride compound which are so called highly unsaturated omega-3-fatty acids and represent essential building blocks for the human body and precursors for prostaglandins and structural elements of membrane lipid synthesis which have an important biologic role. Furthermore these acids have been considered to have an antithrombotic as well as lipid lowering effect. Since isolation of these acids from natural products and the chemical synthesis is very costly, the fish oils are considered relatively inexpensive sources of these essential fatty acids. But the use in fatty emulsions particularly for parenteral purposes mandates that these fish oils are highly purified and meet high quality standards so that with the parenteral administration no health risks and adverse reactions for the patient occur or at least can be avoided. Furthermore desirable that these highly refined fish oils are enriched with omega-3 fatty acid triglycerides. Methods of extracting and refining oils are well known in the art.

The preferred fatty emulsions are characterized by a high content of highly refined fish oil, which is highly enriched beyond the initial content of omega-3 fatty acids and their triglycerin compound as part of this specific procedure. This fish oil contains a minimum of 95 weight percent preferably a 98 weight % of monomeric triglycerides, less than 1 weight percent of oxidized triglycerides, less than 0.2 weight percent preferably less than 0.1 weight percent of trimeric and oligomeric triglycerides and less than 0.8 weight percent preferably even less than 0.5 weight percent of dimeric poly glycerides as well as less than 1.5 weight percent, preferably less than 8 weight percent of unemulsifiable particularly carbohydrates and sterane. The total content of eicosapentaenoic acid and docosahexanoic acid in the triglyceride compound is in the area of 25-50 weight percent preferably 35-50 weights percent as determined by surface percentage in the gas chromatogram. While fish oils usually have a cholesterol content of 4000 to 12000 ppm, the cholesterol content of the fish oils preferred contain less than 2500 ppm preferably less than 1500 ppm.

Preferably, the fish oil enriched omega-3 fatty acid triglyceride components contains primarily eicosapentaenoic and docosahexanoic acid. These can be present in variable ratios as determined by area percentage on gas chromatogram. These mass ratios are dependent on the nature of the fish oil and the degree of enrichment of omega-3 fatty acids. It has been shown that fish oils which contain an eicosapentaenoic acid and docosahexanoic acid in their triglyceride compound mass ratio of 0.5 to 2.6 as determined by surface area on gas chromatogram represent a fat emulsion of excellent quality and therefore this mass ratio is considered ideal and is preferred.

Fish oil is available commercially, for example 10% (wt/wt) fish oil triglycerides can be obtained from Nisshin Flour Milling Co. located in Nisshin, Japan.

To prepare the lipid emulsions in accordance with the present invention, one or more emulsifying agents are mixed with the source of omega-3 fatty acids, e.g. fish oil. Emulsifying agents for this purpose are generally phospholipids of natural, synthetic or semi-synthetic origin. A variety of suitable emulsifying agents are known in the art. Examples of suitable emulsifying agents include, but are not limited to, egg phosphatidylcholine, egg lecithin, L-α-dipalmitoyl phosphatidylcholine (DPPC), DL-α-dipalmitoyl phosphatidylethanolamine (DPPE), and dioleoyl phosphatidylcholine (DOPC). In accordance with the present invention, the total concentration of triglycerides as well as free fatty acids in the emulsifier should be low in order to minimize the contribution to the total oil concentration of the emulsion. In one embodiment of the present invention, the total concentration of triglycerides as well as free fatty acids in the emulsifier is less than about 3.5%.

In one embodiment of the present invention, lecithin is used as the emulsifying agent in the lipid emulsions. Alternatively, egg lecithin can be used as the emulsifying agent. Egg lecithin containing 80-85% phosphatidyl choline and less than about 3.5% of fat can also be used as an emulsifying agent. One skilled in the art will appreciate that other components may be present in the egg lecithin without adversely affecting the emulsifying properties. For example, the egg lecithin may contain one or more of phosphatidyl ethanolamine, lysophosphatidyl choline, lysophosphatidyl ethanolamine, sphingomeylin and other natural components.

The lipid emulsions according to the present invention typically contain between about 0.5% and about 5% (w/v) emulsifying agent. In one embodiment of the present invention, the emulsion contains between about 0.6% and about 2% (w/v) emulsifying agent. In another embodiment, the emulsion contains between about 0.8% and about 1.8% (w/v) emulsifying agent. In another embodiment, the emulsion contains between about 1.0% and about 1.5% (w/v) emulsifying agent. In another embodiment, the emulsion contains between about 1.2% (w/v) emulsifying agent.

The ratio of lecithin to source oil in the emulsion is important in determining the size of the oil globules formed within the emulsion. In one embodiment, the ratio of lecithin to source oil is between about 1:4 and about 1:20. In one embodiment of the present invention, the ratio is between about 1:4 and about 1:18. In another embodiment, the ratio is between about 1:4 and about 1:15. In another embodiment, the ratio is between about 1:4 and about 1:10.

The lipid emulsion in accordance with the present invention can further comprise additional components such as, antioxidants, chelating agents, osmolality modifiers, buffers, neutralization agents and the like that improve the stability, uniformity and/or other properties of the emulsion.

The present invention contemplates addition of one or more antioxidants to the lipid emulsion in order to help prevent the formation of undesirable oxidized fatty acids.

Suitable antioxidants that can be added to the lipid emulsions include, but are not limited to, alpha-tocopherol (vitamin E) and tocotrienols. As is known in the art, tocotrienols are a natural blend of tocotrienols and vitamin E extract concentrated from rice bran oil distillate, which have an antioxidant activity similar to that of alpha-tocopherol (vitamin E). Tocotrienols have a similar structure to vitamin E and contain three double bonds in the carbon side chain of the molecule.

When used, the concentration of antioxidant added to the emulsion is typically between about 0.002 and about 1.0% (w/v). In one embodiment, the concentration of antioxidant used in the emulsion is between about 0.02% and about 0.5% (w/v).

In one embodiment of the present invention, tocotrienols are added to the emulsion as an antioxidant. In another embodiment, about 0.5% (w/v) tocotrienols are added to the emulsion. In still another embodiment, vitamin E is added to the emulsion as an antioxidant. another embodiment, about 0.02% (w/v) vitamin E is added to the emulsion. The emulsion can further comprise a chelating agent to improve the stability of the emulsion and reduce the formation of oxidized fatty acids. Suitable chelating agents are known in the art and are those that are generally recognized as safe (GRAS) compounds. Examples include, but are not limited to, EDTA. In one embodiment of the present invention, the emulsion comprises EDTA. In another embodiment, the emulsion comprises concentrations of EDTA between about 1×10⁻⁶ M and 5×10⁻⁵ M.

Container design is also an important factor when manufacturing fat emulsions. If the emulsion is packaged in glass, it is preferably done in a container that is filled with nitrogen before the actual emulsion is added. After addition of the emulsion, the glass container can be filled again with nitrogen to remove dead space when the cap is affixed. Such nitrogen filling prevents peroxide formation. If the product is packaged in plastic, a DEHP free container that is gas impermeable is preferred. Preferably the container also has the appropriate overwrap to minimize peroxide formation in the lipids as well as leaching of the plasticizer from the container into the product itself. In addition, if plastic is used, it is desirable to have a desiccant in with the bag as well as an indicator that notes if there is a air leak in the overwrap. Preferably the container is also latex free.

An osmolality modifier can also be incorporated into the emulsion to adjust the osmolality of the emulsion to a value suitable for parenteral administration. Amounts and types of osmolality modifiers for use in parenteral emulsions are well-known in the art. An example of a suitable osmolality modifier is glycerol. The concentration of osmolality modifier typically ranges from about 2% to about 5% (w/v). In one embodiment of the present invention, the amount of osmolality modifier added to the emulsion is between about 2% and about 4%. In another embodiment, the amount of osmolality modifier added to the emulsion is between about 2% and about 3%. In another embodiment, about 2.25% (w/v) glycerol is added to the emulsion as an osmolality modifier. The final product should be isotonic so as to allow infusion of the emulsion through either a central or peripheral venous catheter.

One skilled in the art will understand that the pH of the emulsion can be adjusted through the use of buffers or neutralization agents. Emulsions with pH values close to physiological pH or above have been shown to be less prone to fatty acid peroxidation. One skilled in the art will appreciate that the pH of the emulsions can be adjusted through the use of an appropriate base that neutralizes the negative charge on the fatty acids, through the use of an appropriate buffer, or a combination thereof. A variety of bases and buffers are suitable for use with the emulsions of the present invention. One skilled in the art will appreciate that the addition of buffer to the emulsion will affect not only on the final pH, but also the ionic strength of the emulsion. High ionic strengths may negatively impact the zeta potential of the emulsion (i.e. the surface charge of the oil globules) and are, therefore, not desirable.

Selection of an appropriate buffer strength to provide a suitable pH and zeta potential as defined herein is considered to be within the ordinary skills of a worker in the art.

In one embodiment of the present invention, the pH of the emulsion is adjusted using sodium hydroxide. In another embodiment, the pH is adjusted with a buffer. In another embodiment, the buffer is a phosphate buffer. In another embodiment, both sodium hydroxide and a phosphate buffer are added to the emulsion.

The final pH of the emulsion is typically between about 6.0 and about 9.0. In one embodiment of the present invention, the pH of the emulsion is between about 7.0 and about 8.5. In another embodiment, the pH of emulsion is between about 7.0 and about 8.0.

The lipid emulsion can further comprise components for adjusting the stability of the emulsion, for example, amino acids or carbohydrates, such as fructose or glucose. The lipid emulsion can also be formulated to include nutrients such as glucose, amino acids, vitamins, or other parenteral nutritional supplements. The formulation of the lipid emulsion to incorporate a therapeutic agent is also considered to be within the scope of the present invention. A “therapeutic agent” as used herein refers to a physiologically or pharmacologically active substance that produces a localized or systemic effect or effects in animals and refers generally to drugs, nutritional supplements, vitamins, minerals, enzymes, hormones, proteins, polypeptides, antigens and other therapeutically or diagnostically useful compounds.

The lipid emulsions in accordance with the present invention can be prepared by a number of conventional techniques known to those skilled in the art. In general, the core lipid is first mixed with the emulsifier and the antioxidant, if one is being used.

The emulsion is then prepared by slowly adding this oil phase into water with constant agitation. If an osmolality modifier is being used, it is added to the water prior to mixture with the oil phase. The pH can be adjusted at this stage, if necessary, and the final volume adjusted with water, if required.

The size of the oil globules of the emulsion (i.e. the particle size) is an important parameter with respect to therapeutic effects and the quality of the emulsion. Since lipid particles are removed from the systemic circulation in a manner similar to chylomicrons, the size of lipid particles in the emulsion need to remain within or below the size range of the naturally occurring chylomicron, which is 0.4-1.0 um. If the particle size is larger than this, the lipid particles may be deposited in the liver, spleen and lungs resulting in significant fat load following infusion (Rahui C. M., et I al., Am. Hosp. Pharm. 1992, 49:2749-2755). Lipids with small particle sizes disperse better in the emulsion and tend to produce safer and more stable emulsions. Selection of appropriate conditions for the preparation of the emulsions according to the present invention is considered to be within the ordinary skills of a worker in the art.

The above-mentioned components can be present in various mass ratios in the fatty emulsion. The preferred form of the invented fatty emulsion contains 5-45 weight percent of highly refined omega-3 fatty acid enriched fish oil, 1-2 weight percent of emulsifier, 1-2 weight percent of emulsifier stabilizer as well as isotonizing additive, 0.02-0.02 weight percent of co-emulsifier and the rest in water. Especially preferred is a fatty emulsion with 8-35 weight percent of highly enriched omega-3 fatty acid fish oil, 1-1.5 weight percent of emulsifier, 1.5-2.5 weight percent of emulsifying stabilizer and isotonizer add on, 0.03 weight percent of co-emulsifier and the rest in water.

One procedure for manufacturing a fatty emulsion by using purified de-acidified and bleached fish oil with a content of omega-3 fatty acids includes the following: the fish oil is mixed with a fish oil compatible solvent in a weight to volume ration of fish oil to solvent of 1:1 to 1.5 is as follows. The mixture is cooled down to a temperature of −15 to −80 degrees centigrade then filtered of insoluble components, the filtrate is then cautiously separated from the solvent and the soak contained fish oils 2-4 hours steamed at 180-220 degrees Celsius. The absorption of the steamed fish oil in a nonporous solvent and filtering of the obtained solution over a selica gel—untreated with nonpolar solvent, followed by gentle removal of the nonpolar solvent and warming of the obtained highly refined fish oil enriched with omega-3 fatty acids in a nitrogen atmosphere to 50-60 degrees Celsius, filtering through a membrane filter and portion wide addition of sterane to an accurate mixture likewise kept at a controlled temperature of 50-60 degrees Celsius which contained emulsifier stabilizer and isotonization additive and co-emulsifier. Further emulsification of the formed crude emulsion at 60-70 degrees followed by filtering under nitrogen atmosphere through a membrane filter and single or multiple stepped homogenization of the emulsion at 70-85 degrees whereupon the obtained fat emulsion is cooled under nitrogen to a temperature in the range of 5-10 degrees Celsius if necessary adjusted to a pH value of 8.5-8.8 and drawn off into suitable weight under oxygen exclusion.

A preferred fatty emulsion for use in the present invention is Omegaven™ (Fresemius AG).

It is expected that similar therapeutic benefit will result from administration of omega-3 polyunsaturated fatty acid precursors and analogs in the dosage regimens and routes of administration described herein, as compared to the benefit from omega-3 polyunsaturated fatty acid.

### Therapeutic Benefit

Treatment of pathological angiogenesis includes halting disease progression, reversing disease progression, and significant amelioration of disease symptoms. Preventing pathological angiogenesis includes complete prevention of disease onset, slowing of disease onset and/or disease progression following onset resulting from treatment that began prior to onset. Disease progression and onset of neovascularization is measured by the skilled practitioner by any means known and accepted in the field. For ocular neovascularization, treatment will include reduction of ocular neovascularization to an extent that it ameliorates to an appreciable degree the effects of the condition. Treatment generally takes place following diagnosis of the condition or signs of onset of the condition. Preferably, treatment results in complete reversal of the condition, however partial reversal of the condition may also be achieved and is considered of therapeutic benefit to the subject. Such partial reversal can be diagnosed or detected by the skilled practitioner, e.g. by visual examination or functional testing of the subject. Prevention is usually achieved by administration to a subject at risk, prior to diagnosis of the problem or onset, in order to lessen the severity of, or completely prevent or delay disease onset and/or symptoms.

### Conditions, Injuries and Diseases

A variety of conditions, injuries, and diseases produce, or are otherwise associated with, ocular neovascularization. All such conditions, injuries and diseases are suitable for treatment or prevention by the methods described herein. Examples include, without limitation, retinopathy of prematurity, retina vein occlusion, sickle cell retinopathy, choroidal neovascularization, radiation retinopathy, microangiopathy, retinal hyperoxia, diabetic retinopathy, and age related macular degeneration. Subjects diagnosed with or at increased risk for these conditions, diseases or injuries are suitable for the methods described herein.

Subjects suffering from, or at increased risk of, retinopathy of prematurity include infants born pre-term and/or of low birth weight. Preterm refers to the fact that they are born before full term of gestation. Low birth weight means that they weigh at least 10% less than the average weight for their gestational age. Often such low weight infants are not fully developed, especially occularly, and are at high risk for inappropriate development and conditions which arise from inadequate development, including neovascularization. In addition, pre-term infants often receive therapeutic administration of increased oxygen, which is also a known factor in development of neovascularization. These subjects are at high risk for retinopathy of prematurity.

For such patients, administration is preferably as a newborn. In one embodiment, administration begins shortly after birth and is periodic (e.g. at defined intervals), according to a prolonged regimen of administration, until the eye is fully vascularized. In this and all other conditions, diseases and injuries, benefit is expected to result from treatment after onset of the retinopathy as well, as the condition can be ameliorated by treatment for sometime following development of the condition, especially by administration of high-doses of the agent. Added advantage may also be conferred from continued treatment even after full vascularization of the eye. In one embodiment, administration is periodic until the age of one year. One such possible form of oral administration is via supplemented formula. Another such route of administration would be to the mother, e.g. with high doses to the extent required to increase her milk to an effective amount.

Subjects with diabetes are at increased risk for development of diabetic retinopathy and are suitable for the preventative methods described herein. Subjects who have already experienced onset of diabetic retinopathy are suitable for treatment by the methods described herein, and will likely benefit more from high doses of administration (e.g. of omega-3 polyunsaturated fatty acids). Similarly subjects over the age of 55 are at increased risk of ocular neovascularization resulting from age related macular-degeneration, and are suitable for the preventative methods described herein. Subjects who have already experienced onset of the condition will also benefit from treatment described herein, especially from high-doses of administration.

Subjects to receive therapeutic treatment and preventative methods described herein are preferably human. Such treatment will also provide benefit to animals (e.g. mammals) suffering from neovascularization related illnesses described herein, or their equivalents. Animals likely to receive such treatment would be domesticated animals for enjoyment and recreation (e.g. dogs, cats, horses, zoo animals) or livestock, especially grazing livestock (e.g. cattle, sheep, etc.), or any other animal that might benefit from treatment.

Another aspect of the invention relates to kits, or articles for sale, which comprise an agent described herein, formulated for appropriate administration (e.g. a pharmaceutical agent) for the methods described herein. Such kits may further comprise packaging material that comprises a label which indicates one or more of the above recommended routes and/or regimens of administration for treatment of prevention of disease as described herein.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

Other than in the operating examples, or where otherwise indicated, all numbers expressing quantities of ingredients or reaction conditions used herein should be understood as modified in all instances by the term “about.” The term “about” when used in connection with percentages may mean±1%.

In one respect, the present invention relates to the herein described compositions, methods, and respective component(s) thereof, as essential to the invention, yet open to the inclusion of unspecified elements, essential or not (“comprising). In some embodiments, other elements to be included in the description of the composition, method or respective component thereof are limited to those that do not materially affect the basic and novel characteristic(s) of the invention (“consisting essentially of”). This applies equally to steps within a described method as well as compositions and components therein. In other embodiments, the inventions, compositions, methods, and respective components thereof, described herein are intended to be exclusive of any element not deemed an essential element to the component, composition or method (“consisting of”).

All patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

### Examples

### Results and Discussion

Emerging knowledge of the properties of lipid mediators^{2, 5}, as well as retrospective epidemiologic data describing polyunsaturated fatty acid-neovascular age-related macular degeneration relationships, suggests that EPA, DHA, and AA might act in vivo to regulate retinal vaso-obliteration and neovascularization⁵. To further investigate this possibility, the ability of moderate dietary intake of omega-3 polyunsaturated fatty acids or omega-6 polyunsaturated fatty acids to alter retinal angiogenesis was investigated. Mice on a defined isocaloric diet enriched with 2% of total fatty acids from either omega-3 polyunsaturated fatty acids (DHA and EPA) or omega-6 polyunsaturated fatty acid (AA), with their pups nursed with milk reflecting this diet were subjected to the model of oxygen induced retinopathy¹. In addition, the Fat-1 mouse⁹ which converts omega-6 polyunsaturated fatty acids to omega-3 polyunsaturated fatty acids to achieve an elevated omega-3 polyunsaturated fatty acid tissue status genetically was used in the same disease model. This is a transgenic mouse that expresses the gene from C. elegans that allows them to convert omega-6 to omega-3 long chain polyunsaturated fatty acids, resulting in a low omega-6: omega-3 ratio. Tables 1-3 below indicate the Total Composition of Experimental Diets used in the experiments.

TABLE 1
Manufacture Diet Analysis
Summary of Fatty Acid Compositions of Experimental Diets.

| | | | Fatty Acid Profile (% TFA) | | | | | | | |
|---|---|---|---|---|---|---|---|---|---|---|
| Diet | # | General Description | OA | DHA | EPA | ALA | AA | LA | SA | PA |
| D03061805 | 2 | High oleate + ω-3 LCPUFA | 70 | 1 | 1 | ~0 | 0 | 11 | 2 | 6 |
| D04061502 | 4 | High oleate + ω-6 LCPUFA | ~70 | ~0 | ~0 | ~0 | ~2 | ~10 | ~2 | ~5 |
| NIH | 6 | NIH Diet (high in ω-6 precursors) | 24 | 1 | 0.1 | 2 | 0.1 | 55 | 3 | 8 |

Note:
AA = arachidonic acid (20:4ω-6)
ALA = a-linolenic acid (18:3ω-3). Precursor to EPA
DHA = docosahexaenoic acid (22:6ω-3)
EPA = eicosapentaenoic acid (20:5ω-3). Precursor to DHA
LA = linoleic acid (18:2ω-6). Precursor to AA. Main ω-6 in diet
OA = oleic acid (18:1ω-9). The high oleic acid diet was developed with the intention reducing AA precursors.
SA = stearic acid (18:0)
PA = palmitic acid (16:0)
% TFA = percent of total fatty acids

TABLE 2
AIN-93G Rodent Diet and Modified AIN-93G With Different Oils

| Product # | |
|---|---|
| ω-3 LCPUFA | ω-6 LCPUFA |
| Feed | Feed |

| | gm % | kcal % | gm % | kcal % |
|---|---|---|---|---|
| Protein | 20.3 | 19.6 | 20.3 | 19.6 |
| Carbohydrate | 61.0 | 58.8 | 61.0 | 58.8 |
| Fat | 10.0 | 21.7 | 10.0 | 21.7 |
| Total | | 100.0 | | 100.0 |
| kcal/gm | 4.15 | | 4.15 | |

| Ingredient | gm | kcal | gm | kcal |
|---|---|---|---|---|
| Casein, 80 Mesh | 0 | 0 | 0 | 0 |
| Casein, Alcohol | 200 | 800 | 200 | 800 |
| Extracted | | | | |
| L-Cystine | 3 | 12 | 3 | 12 |
| Corn Starch | 150 | 600 | 150 | 600 |
| Maltodextrin 10 | 150 | 600 | 150 | 600 |
| Sucrose | 100 | 400 | 100 | 400 |
| Dextrose | 200 | 800 | 200 | 800 |
| Cellulose, BW200 | 50 | 0 | 50 | 0 |
| Soybean Oil | 0 | 0 | 0 | 0 |
| Safflower Oil, High | 93 | 837 | 93 | 837 |
| Oleic | | | | |
| ROPUFA 30 | 5.7 | 51.3 | 0 | 0 |
| DHASCO (40% DHA) | 1.3 | 11.7 | 0 | 0 |
| ARASCO (40% AA) | 0 | 0 | 7 | 63 |
| t-BHQ | 0 | 0 | 0.014 | 0 |
| Mineral Mix S10022G | 35 | 0 | 35 | 0 |
| Vitamin Mix V10037 | 10 | 40 | 10 | 40 |
| Choline Bitartrate | 2.5 | 0 | 2.5 | 0 |
| Total | 1000.50 | 4152 | 1000.51 | 4152 |

TABLE 3
Fat-1 Feed: AIN-76A Rodent Diet and
Same With 5% or 10% Safflower Oil

| | Product # | |
|---|---|---|
| | Fat-1 Feed | |
| | gm % | kcal % |
| Protein | 21 | 21 |
| Carbohydrate | 59 | 58 |
| Fat | 10 | 22 |
| Total | | 100.0 |
| kcal/gm | 4.13 | |
| Ingredient | gm | kcal |
| Casein | 200 | 800 |
| DL-Methionine | 3 | 12 |
| Corn Starch | 150 | 600 |
| Sucrose | 401 | 1604 |
| Cellulose, BW200 | 50 | 0 |
| Corn Oil | 0 | 0 |
| Safflower Oil | 94 | 846 |
| Mineral Mix S10001 | 35 | 0 |
| Vitamin Mix V10001 | 10 | 40 |
| Choline Bitartrate | 2 | 0 |
| Total | 945 | 3902 |
| C2, Acetic | 0.00 | |
| C4, Butyric | 0.00 | |
| C6, Caproic | 0.00 | |
| C8, Caprylic | 0.00 | |
| C10, Capric | 0.00 | |
| C12, Lauric | 0.00 | |
| C14, Myristic | 0.00 | |
| C14:1, Myristoleic | 0.00 | |
| C16, Palmitic | 6.02 | |
| C16:1, Palmitoleic | 0.00 | |
| C18, Stearic | 2.16 | |
| C18:1, Oleic | 11.28 | |
| C18:2, Linoleic | 73.70 | |
| C18:3, Linolenic | 0.12 | |
| C18:4 | 0.00 | |
| C20, Arachidic | 0.00 | |
| C20:1, | 0.00 | |
| C20:4, Arachidonic | 0.00 | |
| C20:5, | 0.00 | |
| C22, Behenic | 0.00 | |
| C22:1, Erucic | 0.00 | |
| C22:4, Clupanodonic | 0.00 | |
| C22:5 | 0.00 | |
| C22:6, | 0.00 | |
| C24, Lignoceric | 0.00 | |
| Total (gm) | 93.28 | |
| Saturated (g) | 8.2 | |
| Monounsaturated (g) | 11.3 | |
| Polyunsaturated (g) | 73.8 | |
| Saturated (%) | 8.8 | |
| Monounsaturated (%) | 12.1 | |
| Polyunsaturated (%) | 79.1 | |
| total n-6 (gm) | 73.7 | |
| total n-3 (gm) | 0.1 | |

Formulated from J. Nutr. 107: 1340-1348, 1977 and J. Nutr. 110: 1726, 1980 and on Sep. 29, 2003. KangJ02.for.xls

To ensure that retinal composition reflected differences in dietary intake of lipids, the lipid status on pups was first determined by Fast GC/FID analysis. More specifically, the retinal polyunsaturated fatty acid lipid status in pups at postnatal day seventeen (P17) nursed from birth by mothers on a diet enriched in either omega-3 or omega-6 polyunsaturated fatty acids, or in pups expressing the Fat-1 transgene on a high omega-6 polyunsaturated fatty acid diet, verses their wild type controls, was determined by Fast GC/FID analysis. Milk has been previously shown to reflect the lipid profile of the mother's diet^{8, 10}. Both the EPA/DHA enriched diet or expression of the Fat-1 gene in the mother led to an increase in all of the principal omega-3 polyunsaturated fatty acids in the retinas of the milk fed pups including EPA, DPA, omega-3 and DHA (p≦0.005), and a substantial increase in the total omega-3 polyunsaturated fatty acids and a concomitant decrease in the omega-6/omega-3 LC polyunsaturated fatty acid ratio. As indicated below in Table 4, the Fat-1 expressing mice as well as the EPA/DHA supplemented group also had a corresponding decrease in retinal omega-6 polyunsaturated fatty acids including AA, DTA and DPA omega-6 (p≦0.005) and a decrease in the total retinal omega-6 polyunsaturated fatty acids relative to the AA supplemented group, as expected.

TABLE 4
Fatty acyl composition of retinas from P17 pups
Retinal Lipids at P17 (weight % of total fatty acids)

| Fatty Acid Family | ω-6 diet (n = 6) | | ω-3 diet (n = 6) | | Fat-1 WT (n = 6) | | Fat-1 (n = 4) | |
|---|---|---|---|---|---|---|---|---|
| Saturates | | | | | | | | |
| PA (16:0) | 22.53 | (0.14) | 22.81 | (0.24) | 21.40 | (0.05) | 21.83 | (0.88) |
| SA (18:0) | 20.30 | (0.12) | 20.51 | (0.26) | 19.01 | (0.06) | 19.26 | (0.14) |
| Total SFA | 44.85 | (0.21) | 45.39 | (0.43) | 41.84 | (0.43) | 42.31 | (1.14) |
| Monounsaturates | | | | | | | | |
| OA (18:1ω9) | 8.43 | (0.03) | 8.79 | (0.09)‡ | 6.61 | (0.13) | 6.91 | (0.62) |
| VA (18:1ω7) | 2.40 | (0.03) | 2.21 | (0.04)‡ | 2.01 | (0.05) | 1.82 | (0.13) |
| Total MUFA | 11.98 | (0.10) | 12.15 | (0.12) | 9.45 | (0.21) | 9.61 | (0.99) |
| ω-6 Polyunsaturates | | | | | | | | |
| LA (18:2ω6) | 0.74 | (0.01) | 0.90 | (0.04)‡ | 1.65 | (0.04) | 1.76 | (0.09) |
| AA (20:4ω6) | 8.87 | (0.34) | 7.11 | (0.35)‡ | 11.40 | (0.21) | 8.41 | (0.00)‡ |
| DTA (22:4ω6) | 1.25 | (0.15) | 0.57 | (0.09)‡ | 2.25 | (0.03) | 0.85 | (0.05)‡ |
| DPA (22:5ω6) | 4.29 | (0.29) | 0.96 | (0.08)‡ | 4.93 | (0.12) | 0.29 | (0.01)‡ |
| Total ω-6 PUFA | 15.82 | (0.39) | 10.50 | (0.30)‡ | 21.66 | (0.28) | 12.69 | (0.10)‡ |
| ω-3 Polyunsaturates | | | | | | | | |
| ALA (18:3ω3) | 0.03 | (0.003) | 0.03 | (0.01) | 0.01 | (0.00) | 0.03 | (0.01)‡ |
| EPA (20:5ω3) | 0.02 | (0.0002) | 0.25 | (0.02)‡ | 0.00 | (0.00) | 0.52 | (0.01)‡ |
| DPA (22:5ω3) | 0.17 | (0.01) | 0.47 | (0.03)‡ | 0.15 | (0.01) | 0.76 | (0.01)‡ |
| DHA (22:6ω3) | 12.65 | (0.93) | 17.92 | (1.07)‡ | 17.58 | (0.22) | 26.58 | (0.39)‡ |
| Total ω-3 PUFA | 12.87 | (0.93) | 18.68 | (1.07)‡ | 17.74 | (0.22) | 27.93 | (0.37)‡ |
| DHA/DPAω6 | 3.08 | (0.40) | 19.63 | (2.32)‡ | 3.57 | (0.13) | 92.36 | (5.14)‡ |

| | | | | |
|---|---|---|---|---|
| ω-6/ω-3 ratio | 1.23 | 0.56 | 1.22 | 0.45 |

*Retinal lipids were compared in pups fed by dams on an ω-3 or ω-6 PUFA diet or in mice expressing the Fat-1 gene and their WT controls on a high ω-6 PUFA diet. Statistical significance of these comparisons is represented in the ω-3 diet column:
‡p ≦ 0.005 (standard deviation).
PA, palmitic acid; SA, stearic acid; SFA, saturated fatty acids; OA, oleic acid; VA, vaccenic acid; MUFA, monounsaturated fatty acids; LA, linoleic acid; AA, arachidonic acid; DTA, docosatetraenoic acid; DPA, docosapentaenoic acid; PUFA, polyunsaturated fatty acids; ALA, alpha-linolenic acid; EPA, eicosapentaenoic acid; DHA, docosahexaenoic acid.

### Elevated Levels of Omega-3 Polyunsaturated Fatty Acids Result in Decreased Vaso-Obliteration and Retinopathy in Mice

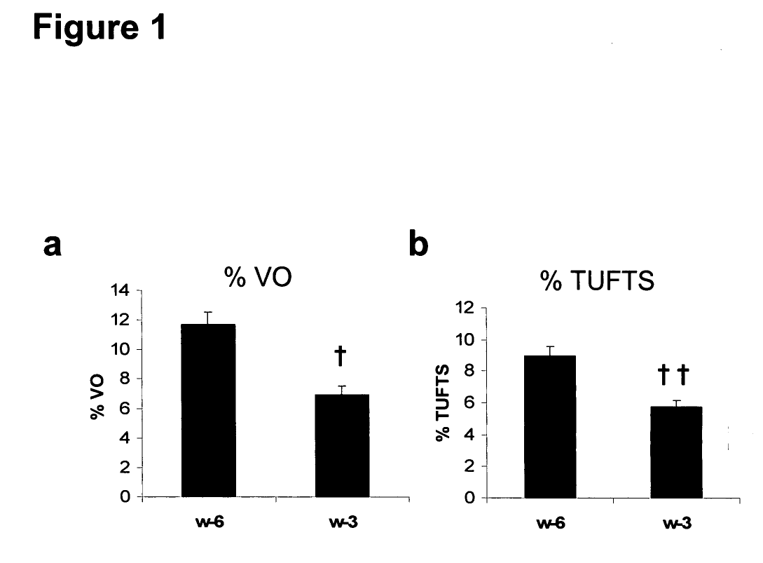

The effects against pathological angiogenesis of dietary modifications in omega-3 or omega-6 polyunsaturated fatty acids was first analyzed. Mice subjected to conditions to generate oxygen induced retinopathy as per the model¹ were fed from dams on either the moderately enriched omega-6 polyunsaturated fatty acid diet, or on an omega-3 polyunsaturated fatty acid diet, and their retina were examined at P17. The retinal vasculature of the mice were stained with lectin-FITC and compared. The omega-6 recipient pups had significantly more extensive vaso-obliteration and neovascularization than the omega-3 recipient pups. (omega-6 n=1, and omega-3 n=27). The mice which received milk generated from a diet of moderately enriched omega-6 polyunsaturated fatty acid diet had a vaso-obliterated area of 11.7±3.2% (mean±S.E.M.) of total retinal area whereas the area of vaso-obliteration in mice on an omega-3 polyunsaturated fatty acid diet was 6.9±3.2% (†p≦0.0001, FIG. 1a). At P17 there was a significant protective effect from pathologic neovascularization in pups fed from dams on an omega-3 polyunsaturated fatty acid enriched diet (FIG. 1b). The mean neovascular growth in omega-3 polyunsaturated fatty acid fed mice was 5.7±2.0% of the total retinal area, compared to 9.0±2.3% (††p≦0.0001, FIG. 1b) for those on an omega-6 polyunsaturated fatty acid diet.

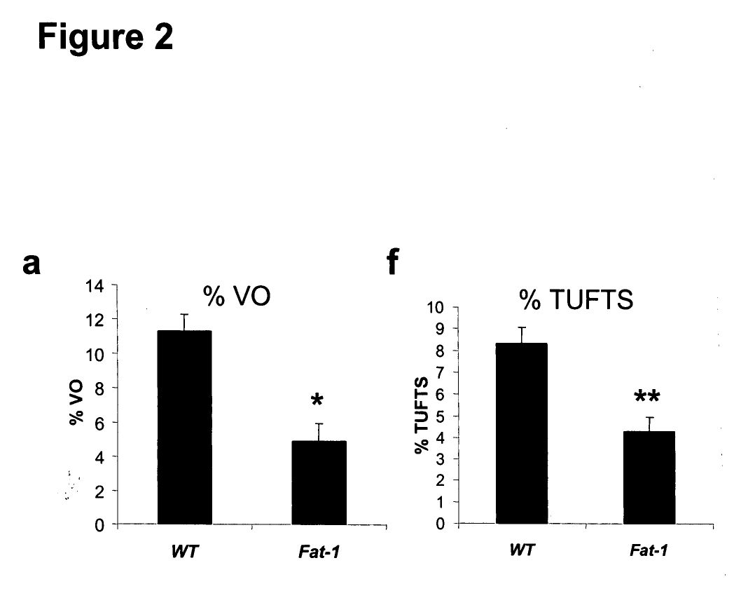

Mice expressing the fat-1 transgene which converts omega-6 to omega-3 polyunsaturated fatty acid were then used experimentally to validate effects on retinal neovascularization through manipulation of polyunsaturated fatty acids in diet. These mice have an elevated omega-3 polyunsaturated fatty acid and reduced omega-6 polyunsaturated fatty acid tissue level when fed an omega-3 polyunsaturated fatty acid deficient, omega-6 polyunsaturated fatty acid replete diet⁹. To evaluate the effect of polyunsaturated fatty acid changes on vessel survival and re-growth, Fat-1 mice and wild type controls were subjected to 75% oxygen from P7 to P12 to induce vessel loss¹ and their retinas examined as above. At P17 wild type mice lacking the fat-1 gene had extensive oxygen-induced vaso-obliteration (11.3±4.5% of total retinal area) as compared to fat-1 expressing mice (4.9±4.3%, *p<0.001; FIG. 2a). Hypoxia-induced retinal neovascularization is maximal in the model at P17¹. Following the induction of retinopathy, wild type mice at P17 had significantly more severe retinal neovascularization (8.3±3.3% of total retinal area) than did the fat-1 homozygotes (4.3±2.6% **p<0.001; FIG. 2b). Note that the Fat-1 mice enjoyed a higher level of protection from pathology than the diet modified mice, (comparing the levels of pathology of the omega-6 diet mice vs. the omega-3 diet mice, and the levels of pathology of the WT vs. the Fat-1 mice, shown in FIGS. 1a and b, FIGS. 2a and b, and this is thought to result from the even higher levels of retinal omega-3 polyunsaturated fatty acids. Without being bound by theory, it is thought that the increased levels of retinal omega-3 polyunsaturated fatty acid resulted from a decreased ratio of total omega-6: omega-3.

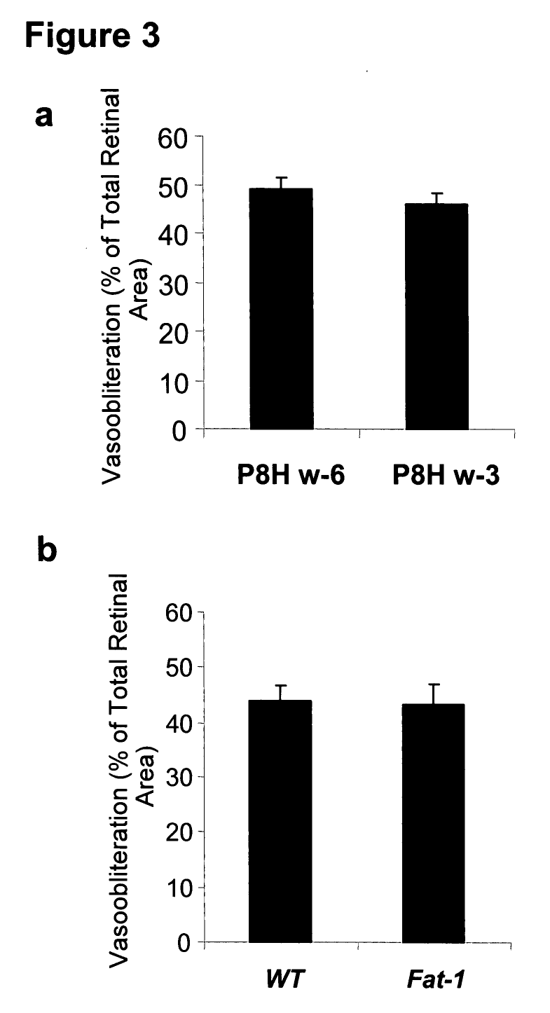

The results of the experiments described above indicate that elevation of omega-3 polyunsaturated fatty acid protected against retinal vaso-obliteration and retinal neovascularization at P17. Two possibilities regarding the protective mechanism exist; elevated omega-3 polyunsaturated fatty acid may have increased vessel re-growth or may have decreased oxygen-induced vessel loss. To assess the contribution of oxygen-induced vessel loss mice either on a omega-3 polyunsaturated fatty acid or omega-6 polyunsaturated fatty acid diet, or fat-1 mice and their wild-type controls subjected to oxygen induced retinopathy as described herein were assessed at P8 during hyperoxia exposure. The assessment revealed that elevated omega-3 polyunsaturated fatty acid by dietary intake or genetically in fat-1 mice did not protect against oxygen-induced vessel loss at P8 (FIG. 3a, b). These results indicate that the protective effect exerted by omega-3 polyunsaturated fatty acids against retinal neovascularization is mediated by enhanced vessel regrowth rather than through suppression of oxygen-induced vessel loss.

ResolvinD1, ResolvinE1 and NeuroprotectinD1, Derived from Omega-3 Polyunsaturated Fatty Acids are Potent Protectors Against Retinopathy with Reduction in Vaso-Obliteration and Neovascularization

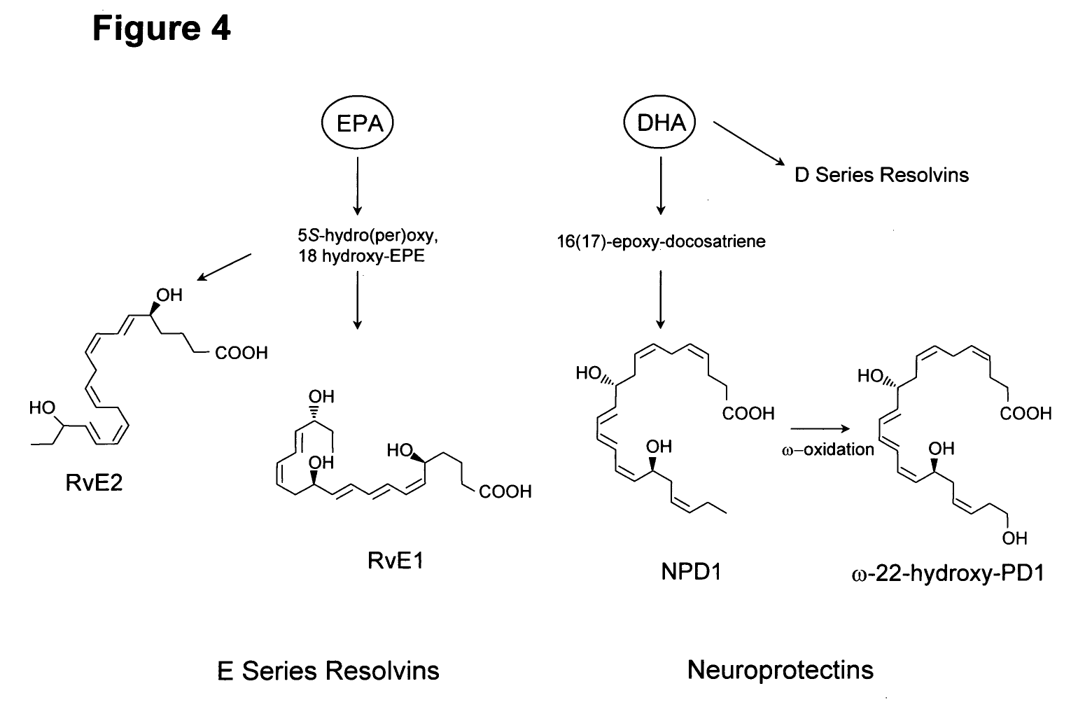

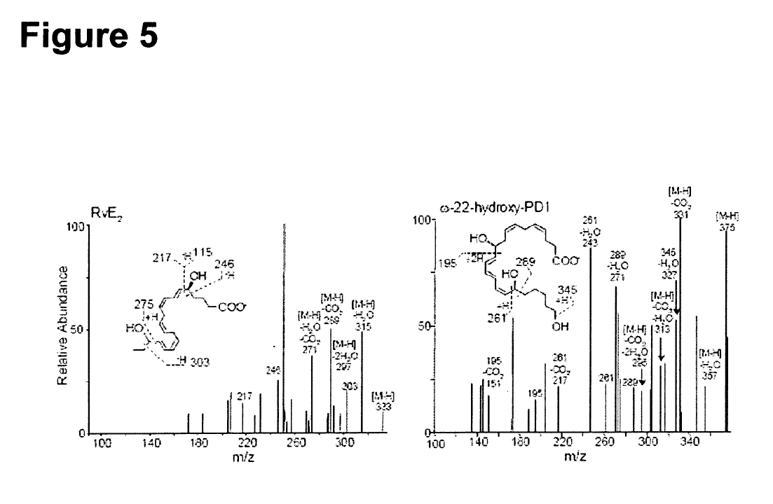

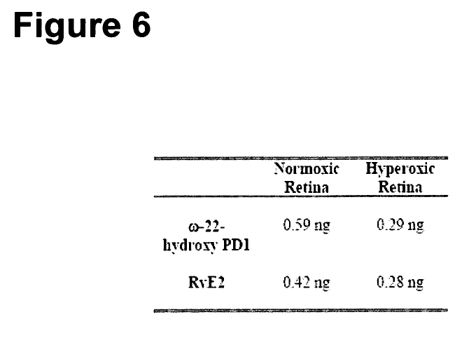

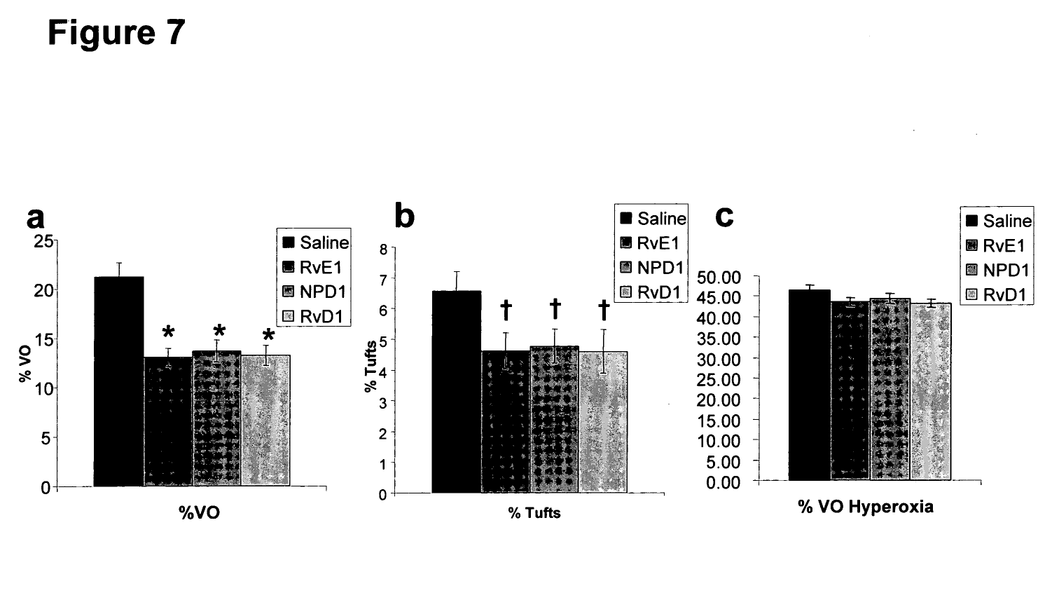

The resolvins (resolution phase interaction products) and neuroprotectins (including neuroprotectin D1, also known as protectin D1) are omega-3 polyunsaturated fatty acid bioactive products derived from EPA and DHA (FIG. 4) that were first identified in resolving inflammatory exudates in tissues enriched with DHA¹¹. The contribution to regulation of angiogenesis by resolvins and neuroprotectins has yet to be investigated¹¹. Retinas of pups fed from dams on diets rich in omega-3 or omega-6 polyunsaturated fatty acids were analyzed for the presence of resolvins and neuroprotectins. In the retinas of the mice pups fed from dams on an omega-6 polyunsaturated fatty acid diet, resolvin or neuroprotectin family members could not be detected. Conversely, in mice fed from dams given the omega-3 polyunsaturated fatty acid diet, omega-22-hydroxy-PD1 and resolvinE2 (RvE2) were identified, each of which are biosynthetic pathway markers (FIG. 4) formed in the biosynthesis of neuroprotectinD1 (NPD1) and resolvinE1 (RvE1) respectively (FIGS. 5 and 6)^{12, 13}. To determine if these bioactive products mediated protective activities of omega-3 polyunsaturated fatty acids against retinopathy, the role of resolvin family members, resolvinD1 (RvD1) and RvE1 as well as the neuroprotectin NPD1, in vessel loss and neovascularisation was assessed in the oxygen-induced retinopathy model. A very low dose of NPD1, RvD1, RvE1 (10 ng/day, comparable to levels found in the omega-3 polyunsaturated fatty acid-treated retinas in vivo (FIG. 6)) or saline was administered intraperitoneally (i.p.) from P6-P17 in mice with oxygen-induced retinopathy. RvD1, RvE1 and NPD1 conferred significant protection from vaso-obliteration, compared to saline-injected controls (*p≦0.0001, FIG. 7a). In addition, less neovascularization at P17 in RvD1, RvE1 and NPD1 treated mice was observed compared to saline controls (†p≦0.03, FIG. 7b). To determine if the decrease in vaso-obliteration of RvD1, RvE1 and NPD1 treated mice was caused by enhanced vessel regrowth or prevention of vessel loss, mice were treated earlier during oxygen-induced vaso-obliteration from P5-P8. No apparent differences in vessel loss was observed between RvD1, RvE1 or NPD1 treated mice and the saline-injected control mice (FIG. 7c), indicating that these compounds confer their protective actions against retinopathy via enhanced vessel re-growth, and not via the suppression of vessel loss. These central findings are concordant with those from the dietary polyunsaturated fatty acid results presented above. Together they suggest that the effect of omega-3 polyunsaturated fatty acids on retinal neovascularization is consistent with actions, at least in part, with the biosynthesis of their potent bioactive mediators NPD1 and RvE1.

Diets Rich in Omega-6 Polyunsaturated Fatty Acid Induce Increased Retinal TNF-α Expression and Retinopathy which is Reversed by Blocking TNF-α.

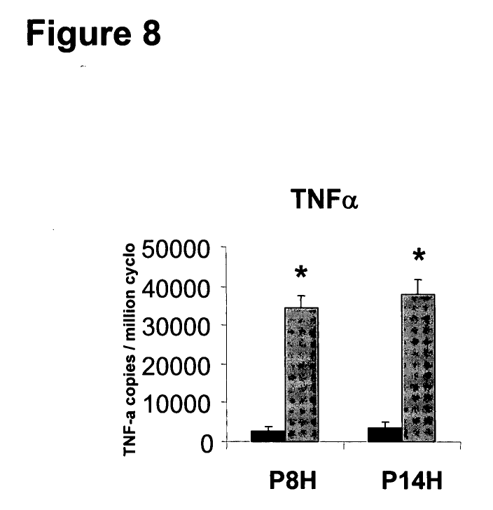

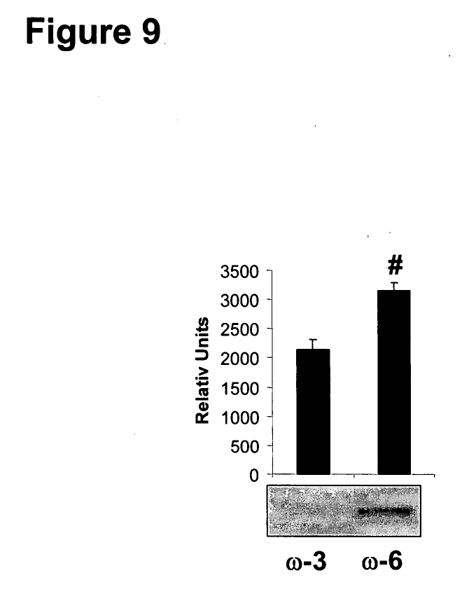

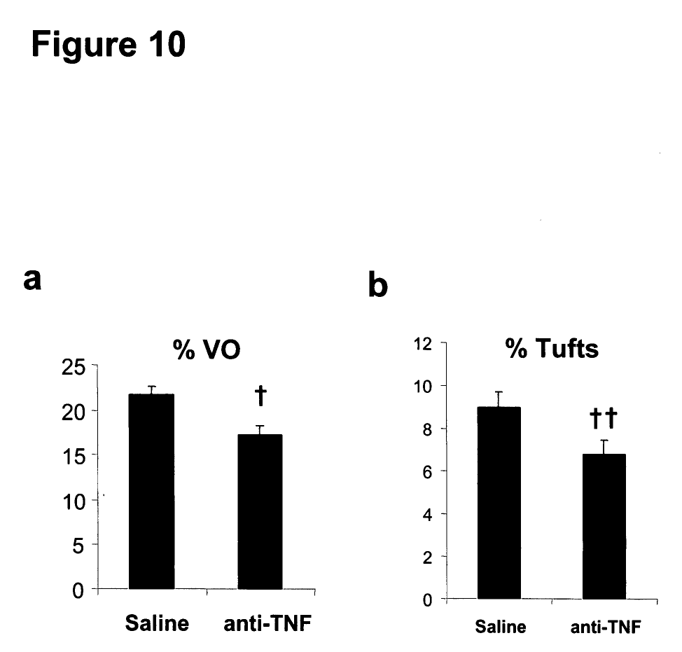

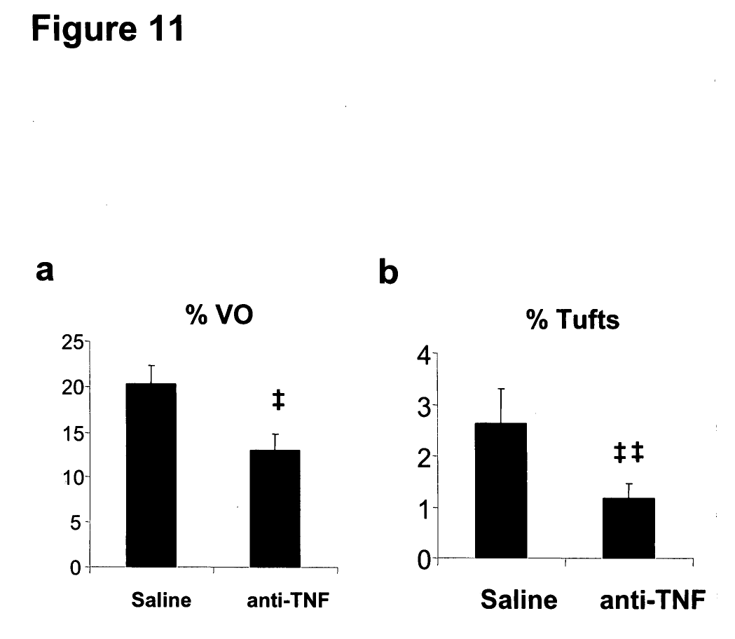

NPD1, RvD1 and RvE1 each significantly reduce TNF-α mRNA expression levels in inflammatory models^{14, 15 and 16}. In addition, mice lacking TNF-α are protected from oxygen-induced retinopathy¹⁷. Given the above findings, the role of dietary intake of either omega-3 or omega-6 polyunsaturated fatty acids on retinal expression of TNF-α was explored by analysis of levels of TNF-α mRNA in pups fed from dams fed on the omega-3 diet and on the omega-6 diet following oxygen induction of retinopathy. The omega-3 polyunsaturated fatty acid diet potently suppresses TNF-α mRNA expression by ˜90% at both P8 (hyperoxia) and P14 (hypoxia) compared to an omega-6 polyunsaturated fatty acid diet (*p≦0.0001, FIG. 8). In addition, retinal levels of TNF-α protein were significantly reduced in pups fed by dams on an omega-3 polyunsaturated fatty acid diet relative to those fed by dams on an omega-6 polyunsaturated fatty acid diet (#p≦0.001, FIG. 9). To further analyze the role of omega-6 polyunsaturated fatty acid on TNF-α during pathological neovascularization, TNF-α receptor fusion protein (etanercept) was injected i.p. to lower systemic TNF-α levels in omega-6 polyunsaturated fatty acid fed mice. Treatment with the TNF-α receptor fusion protein significantly protected pups on the omega-6 polyunsaturated fatty acid diet (with elevated levels of TNF-α) from vessel loss (†p≦0.001, FIG. 10a) as well as from pathologic neovascularization (††p≦0.05, FIG. 10b). This data suggests that the protective effect of omega-3 versus omega-6 polyunsaturated fatty acid diet was consistent with a relative increase in TNF-α in the pups of the omega-6 polyunsaturated fatty acid diet group. Intraocular injections of the TNF-α receptor fusion protein versus saline injection in the fellow eye also significantly reduced vaso-obliteration (‡p≦0.003, FIG. 11a) and also suppressed retinal neovascularization (‡‡p≦0.03, FIG. 11b) in pups in the omega-6 polyunsaturated fatty acid diet group. It should be noted that any intraocular injections (control or treatment) greatly reduce neovascularization.

The omega-3 (DHA, EPA) and omega-6 (AA) polyunsaturated fatty acids significantly influence vascular pathology. EPA and DHA and their potent bioactive products NPD1 and RvE1 at physiological levels promote vessel re-growth after vascular loss and injury as well as reduced pathologic neovascularization. Mice on an omega-6 polyunsaturated fatty acid diet have elevated levels of TNF-α which increases retinopathy. These effects on angiogenesis are important for a number of diseases such as diabetic retinopathy and retinopathy of prematurity as well as other pathologies where vascular loss precipitates disease. The omega-3 polyunsaturated fatty acid suppressive effect on retinopathy in the mouse eye is comparable in magnitude to anti-VEGF treatment¹⁸, and is likely to be additive to anti-VEGF therapy since VEGF is not significantly suppressed with the omega-3 polyunsaturated fatty acid diet.

These results suggest that enriching the sources of omega-3 polyunsaturated fatty acid may be an effective therapeutic approach to help prevent proliferative retinopathy. The resolvin RvE1 and the neuroprotectin NPD1 are potent anti-inflammatory and pro-resolving mediators¹⁹. The present studies establish the first results indicating that these novel mediators are also potent regulators of pathologic angiogenesis. Currently anti-VEGF treatment is approved for age-related macular degeneration and is likely to be beneficial in retinopathy as well²⁰, but these drugs involve repetitive invasive intra-ocular injections. If supplementing sources of DHA and EPA or their bioactive mediators are found to be as effective in ameliorating retinal vascular disease in humans as demonstrated in the present studies, this cost effective intervention could benefit millions of patients.

Late administration of Dietary Omega-3 Long Chain Polyunsaturated Fatty Acids (LCPUFAs)

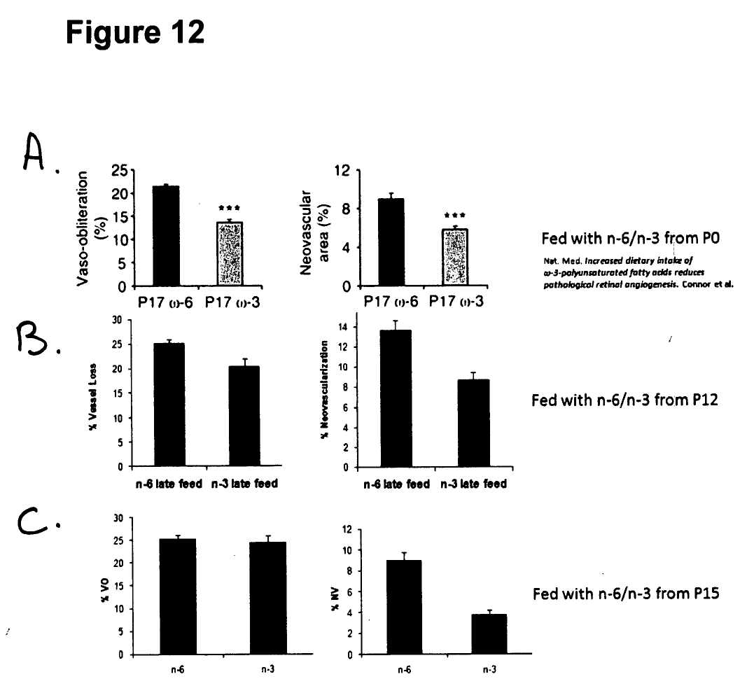

Litters of mice were subjected to the oxygen induced retinopathy model. Briefly, mice (pups with mothers) on normal chow were placed in 75% oxygen at postnatal day 7, and kept at 75% oxygen for five days. Mice were then returned to room air. During this room air phase (P12-P17) the retina becomes hypoxic due to the vessel regression that occurred while mice were in hyperoxia. Here mice were given either an omega-3 or omega-6 LCPUFA diet. At p12 (once out of oxygen, after vessel loss) or at P15 (a few days later as retinopathy was setting in). In mice given omega-3 LCPUFAs at P12 significant protective effect from both vessel regrowth (p=0.01) as well as from pathological neovascularization (p=0.0005) was observed (FIG. 12 (b)). Significantly, even in mice given omega-3 LCPUFAs in the late stages of retinopathy (P15), were protected from pathological neovascularization (p=0.00001) FIG. 12(c)). This data indicates that omega-3 LCPUFAs are protective against pathological angiogenesis and pathologies associated with/arising from angiogenesis even in the late stages of retinopathy.

### Materials and Methods

Animals. These studies adhered to the ARVO Statement for the Use of Animals in Ophthalmic and Vision Research. Fat-1 transgenic mice contain a humanized fat-1 cDNA driven by the cytomegalovirus enhancer and a chicken β-actin promoter⁹. Fat-1 and control dams were fed a defined diet with elevated omega-6 polyunsaturated fatty acids. For diet studies C57Bl/6 mothers at delivery (unless otherwise indicated for FIG. 12) were fed a defined rodent diet with 10% (w/w) safflower oil containing 2% omega-6 polyunsaturated fatty acids (AA) and no omega-3 polyunsaturated fatty acids or the same defined diet except for 2% omega-3 polyunsaturated fatty acids (DHA and EPA) and no omega-6 polyunsaturated fatty acids (AA). (Table 1) Diets were stable over time and with oxygen exposure (Table 2), unless otherwise indicated.

O₂-induced retinopathy (vessel degeneration, re-growth and pathological neovascularization). To induce vessel loss, postnatal day 7 (P7) mice with their nursing mother were exposed to 75% oxygen for times ranging from 24 hours to 5 days¹. To evaluate vaso-obliteration following 24 hours of oxygen exposure, P8 mice were anesthetized at with Avertin (Sigma) and perfused with 50 μl of 120 mg/ml FITC-dextran (2×10⁶ molecular weight, FD2000S-5G, Sigma) in saline through the left ventricle²¹. Eyes were enucleated and fixed in 4% paraformaldehyde for 2 h at 4° C. Retinas were isolated and whole-mounted with SlowFade Antifade reagent (S2828, Molecular Probes) onto polylysin-coated slides with the photoreceptor side up. Retinas were examined with a fluorescence microscope (Olympus, Tokyo), digitized images using a three-charge-coupled device color video camera (DX-950P, Sony), and processed with NORTHERN ECLIPSE software (Empix Imaging, Toronto). Retinal neovascularization was evaluated 5 days after oxygen exposure (P7-P12) at P17 when the neovascular response is greatest. P17 mice were given a lethal dose of Avertin (Sigma) and their eyes were enucleated and fixed in 4% paraformaldehyde for 2 h at 4° C. Retinas were isolated and stained overnight with fluoresceinated Griffonia Bandereiraea Simplicifolia Isolectin B4 (Alexa Fluor 488-I21411 or Alexa Fluor 594-I21413, Molecular Probes) in 1 mM CaCl₂ in PBS. Following 2 hours of washes, retinas were whole-mounted with glycerol-gelatin (Sigma) onto polylysin-coated slides with the photoreceptor side up and imaged with a confocal microscope.

Quantification of vaso-obliteration and retinal neovascularization. Images of each of 4 quadrants of whole-mounted retina were taken at 5× magnification and imported into Adobe Photoshop. Retinal segments were merged to produce an image of the entire retina. Vaso-obliteration and neovascular tuft formation were quantified by comparing the number of pixels in the affected areas with the total number of pixels in the retina. Percentages of vaso-obliteration and neovascularization from mouse retinas were compared with values for retinas from age-matched control mice with identical oxygen conditions^{17,22}. Evaluation was done blind to the identity of the sample.

Resolvins and Neurorotectins. C57Bl/6 nursing mothers were fed a diet rich in either omega-6 or omega-3 polyunsaturated fatty acids from birth. To induce proliferative retinopathy in the pups, mice were exposed to 75% oxygen from P7 to P12. Retinas collected at P17 from omega-6 or omega-3 polyunsaturated fatty acid fed dams and exposed to high oxygen or room air conditions were analyzed to determine lipid mediator profiles. Polyunsaturated fatty acid derived products were extracted, identified, and quantified using a deuterium-labelled internal standard and MS-MS based mediator informatics²³. Results were obtained from retinas of six mice, each from a separate litter. Treatments with synthetic RvD1 (7S,8R,17S-trihydroxy-docosa-4Z,9E,11E,13Z,15E,19Z-hexaenoic acid), RvE1 (5S,12R,18R-trihydroxy-6Z,8E,10E,14Z,16E-eicosapentaenoic acid) and Neuroprotectin-D1 (10R,17S dihydroxy-docosa-4Z,7Z,11E,13E,15Z,19z-hexaneoic acid) were performed in 4 litters of mice. RvD1, RvE1 and NPD1 were prepared by organic synthesis according to published procedures matching physical and biological criteria^{14, 24, 25}. The pups were injected i.p. daily from P6-P17 (before, during, and after exposure to 75% oxygen) with 10 ng of either RvD1 (n=14), RvE1 (n=10) or NPD1 (n=14) or Saline/EtOH (n=14) vehicle. Following retinal staining and whole-mounting, neovascularization and vaso-obliteration were quantified at P17. Vaso-obliteration was also assessed at P8 (after 24 hours of 75% oxygen exposure) in pups given 10 ng of RvD1 (n=7), RvE1 (n=9), NPD1 (n=7), or saline/EtOH vehicle injections (n=6) i.p. from P5-P8. Vaso-obliteration was quantified on retinal whole-mounts following FITC-dextran intracardiac perfusion and fixation as above.

Quantitative analysis of gene expression (quantitative real-time PCR). PCR primers targeting Fat-1 and TNF-α and an unchanging control gene, cyclophilin were designed by using Primer Express software (Applied BioSystems, Foster City, Calif.). Three methods were used to analyze primer and probe sequences for specificity of gene detection. First, only primer and probe sequences that specifically detect the sequence of choice, as determined by means of the NCBI Blast module, were used. Second, amplicons generated during the PCR reaction were analyzed using the first derivative primer melting curve software supplied by Applied BioSystems. This analysis determined the presence of amplicons on the basis of their specific melting point temperatures. Third, amplicons generated during the PCR reaction were gel purified and sequenced (Children's Hospital Core Sequencing Facility, Boston, Mass.) to confirm the selection of the desired sequence. Quantitative analysis of gene expression was generated using an ABI Prism 7700 Sequence Detection System (TaqMan) and the SYBR Green master mix kit. Retinas were isolated from 6 mice per group and retinal RNA was isolated and converted to cDNA. (n=6 retinas per time point)

Lipid Extraction and Fatty Acid Analysis. Retinal samples containing 1 retina each were stored in buffered saline (10 mM Tris, 60 mM KCl, 30 mM NaCl, 2 mM Cl₂, 50 μM DTPA, 1.5 mM DTT and 1.5 ml/L aprotinin; adjusted to pH 8.0) at −80° C. until just prior to analysis. The samples were thawed and lipid extracted as previously described by Bligh and Dyer²⁶. Briefly, methanol containing 40 μg/ml butylated hydroxytoluene as an antioxidant was added to the retinal samples and chloroform was added to adjust the solvent ratios to 2:2:1.8 methanol/chloroform/water. The internal standard was 22:3n3 methyl ester (1.5 ug/mg tissue). Samples were homogenized for 30 sec using an Omni TH hand-held homogenizer. The homogenizer probe tip was cleaned in a solution containing chloroform/methanol/water between samples. Samples were vortexed for 1 min and centrifuged at 4° C. for 7 min at 3500 rpm (approx 2000×g) using a Sorvall RT7+ table-top centrifuge. The lower layer was collected. This procedure was repeated two times and the extracts pooled. The chloroform layer was then evaporated and then the samples were redissolved in chloroform. Half of the total lipid extract was taken for transmethylation according to the method of Morrison and Smith²⁷ as modified by Salem et al²⁸.

Methyl esters were quantified on a model 6890 series gas chromatograph (Agilent Technologies, Palo Alto, Calif.) using a FAST-GC method as described by Masood et al²⁹ using a 1 μl injection at a 25:1 split ratio. Tissue fatty acid methyl ester peak identification was performed by comparison to the peak retention times of a 28 component methyl ester standard (462, Nu-Chek Prep, Elysian, Minn.).

TNF-α receptor treatment. Intraperitoneal injections of a soluble TNF-α receptor (etanercept) (500 μg/mouse) were given at P7, P12, P14 and P16 to mice raised on and omega-6 rich diet as previously described³⁰. Retinas were isolated and stained with lectin-rhodamine at P17 to evaluate vaso-obliteration and retinal neovascularization. (n=6 mice per time point)

Intraocular injections. Mice with ischemic retinopathy were given an intravitreous injection of either etanercept (right eye) or a balanced salt solution (Alcon, left eye) on P12 after five days of 75% oxygen treatment. Each mouse received 0.5 microliters containing 12.5 μg of etanercept or saline (fellow eye). Injections were performed by inserting an Exmire microsyringe (MS-NE05, ITO Corp. Fuji, Japan) into the vitreous 1 mm posterior to the corneal limbus. Mice were anesthetized and their pupils were dilated with 1% tropicamide. Insertion and infusion were directly viewed through an operating microscope, taking care not to injure the lens or the retina. Retinal flatmounts of mice were analyzed 5 days post-injection at P17.

Western Blotting. Mice on an omega-3 or omega-6 polyunsaturated fatty acid diet were sacrificed and retinas were collected at P14. Retinas were homogenized and sonicated in 0.05 mM KPi buffer containing an array of phosphatase and protease inhibitors. Samples were normalized using a BSA assay (Pierce) and 50 μg of retinal lysate was loaded on a SDS-PAGE gel and then electroblotted onto PVDF membrane. The primary antibodies was rat anti-mouse TNF-α (Abcam), followed by an incubation with horseradish peroxidase-conjugated goat anti-rat IgG (Amersham) as the secondary antibodies. Antibodies were used according to manufactures recommendations. The primary antibody was applied overnight in 5% BSA at 4° C. Four mice per diet were used. Densitometry was analyzed using ImageJ.

Statistical Analysis. Results are presented as mean±SEM. for animal studies and mean±SD unless otherwise noted. ANOVA with α=0.05 was used for processing the data. A two-sample t test was used as a posttest unless otherwise indicated.

### REFERENCES

All references cited herein are incorporated by reference.
1. Smith, L. E. et al. Oxygen-induced retinopathy in the mouse. Invest Opthalmol Vis Sci 35, 101-11 (1994).
2. Kermorvant-Duchemin, E. et al. Trans-arachidonic acids generated during nitrative stress induce a thrombospondin-1-dependent microvascular degeneration. Nat Med 11, 1339-45 (2005).
3. Fierro, I. M., Kutok, J. L. & Serhan, C. N. Novel lipid mediator regulators of endothelial cell proliferation and migration: aspirin-triggered-15R-lipoxin A(4) and lipoxin A(4). J Pharmacol Exp Ther 300, 385-92 (2002).
4. Fliesler, S. J. & Anderson, R. E. Chemistry and metabolism of lipids in the vertebrate retina. Prog Lipid Res 22, 79-131 (1983).
5. SanGiovanni, J. P. & Chew, E. Y. The role of omega-3 long-chain polyunsaturated fatty acids in health and disease of the retina. Prog Retin Eye Res 24, 87-138 (2005).
6. Calder, P. C. Polyunsaturated fatty acids and inflammation. Prostaglandins Leukot Essent Fatty Acids (2006).
7. Salem, N., Jr., Litman, B., Kim, H. Y. & Gawrisch, K. Mechanisms of action of docosahexaenoic acid in the nervous system. Lipids 36, 945-59 (2001).
8. Serhan, C. N. & Savill, J. Resolution of inflammation: the beginning programs the end. Nat Immunol 6, 1191-7 (2005).
9. Kang, J. X., Wang, J., Wu, L. & Kang, Z. B. Transgenic mice: fat-1 mice convert n-6 to n-3 fatty acids. Nature 427, 504 (2004).
10. Moriguchi, T. et al. Effects of an n-3-deficient diet on brain, retina, and liver fatty acyl composition in artificially reared rats. J Lipid Res 45, 1437-45 (2004).
11. Serhan, C. N., Arita, M., Hong, S. & Gotlinger, K. Resolvins, docosatrienes, and neuroprotectins, novel omega-3-derived mediators, and their endogenous aspirin-triggered epimers. Lipids 39, 1125-32 (2004).
12. Hong, S., Gronert, K., Devchand, P. R., Moussignac, R. L. & Serhan, C. N. Novel docosatrienes and 17S-resolvins generated from docosahexaenoic acid in murine brain, human blood, and glial cells. Autacoids in anti-inflammation. J Biol Chem 278, 14677-87 (2003).
13. Tjonahen, E., Oh, S. F., Siegelman, J., Elangovan, S., Percarpio, K. B., Hong, S., Serhan, C. N. Resolvin E2: Identification and anti-inflammatory actions of a novel w-3 eicosapentaenoic acid-derived mediator. Cell. In Press. (2006).
14. Duffield, J. S. et al. Resolvin d series and protectin d1 mitigate acute kidney injury. J Immunol 177, 5902-11 (2006).
15. Mukherjee, P. K., Marcheselli, V. L., Serhan, C. N. & Bazan, N. G. Neuroprotectin D1: a docosahexaenoic acid-derived docosatriene protects human retinal pigment epithelial cells from oxidative stress. Proc Natl Acad Sci USA 101, 8491-6 (2004).
16. Arita, M. et al. Resolvin E1, an endogenous lipid mediator derived from omega-3 eicosapentaenoic acid, protects against 2,4,6-trinitrobenzene sulfonic acid-induced colitis. Proc Natl Acad Sci USA 102, 7671-6 (2005).
17. Gardiner, T. A. et al. Inhibition of tumor necrosis factor-alpha improves physiological angiogenesis and reduces pathological neovascularization in ischemic retinopathy. Am J Pathol 166, 637-44 (2005).
18. Aiello, L. P. et al. Suppression of retinal neovascularization in vivo by inhibition of vascular endothelial growth factor (VEGF) using soluble VEGF-receptor chimeric proteins. Proc Natl Acad Sci USA 92, 10457-61 (1995).
19. Bannenberg, G. L. et al. Molecular circuits of resolution: formation and actions of resolvins and protectins. J Immunol 174, 4345-55 (2005).
20. Michels, S., Schmidt-Erfurth, U. & Rosenfeld, P. J. Promising new treatments for neovascular age-related macular degeneration. Expert Opin Investig Drugs 15, 779-93 (2006).
21. D'Amato, R., Wesolowski, E. & Smith, L. E. Microscopic visualization of the retina by angiography with high-molecular-weight fluorescein-labeled dextrans in the mouse. Microvasc Res 46, 135-42 (1993).
22. Banin, E. et al. T2-TrpRS inhibits preretinal neovascularization and enhances physiological vascular regrowth in OIR as assessed by a new method of quantification. Invest Opthalmol Vis Sci 47, 2125-34 (2006).
23. Lu, Y., Hong, S., Gotlinger, K. & Serhan, C. N. Lipid mediator informatics and proteomics in inflammation resolution. Scientific World Journal 6, 589-614 (2006).
24. Arita, M. et al. Stereochemical assignment, antiinflammatory properties, and receptor for the omega-3 lipid mediator resolvin E1. J Exp Med 201, 713-22 (2005).
25. Serhan, C. N. et al. Anti-inflammatory actions of neuroprotectin D1/protectin D1 and its natural stereoisomers: assignments of dihydroxy-containing docosatrienes. J Immunol 176, 1848-59 (2006).
26. Bligh, E. G. & Dyer, W. J. A rapid method of total lipid extraction and purification. Can J Biochem Physiol 37, 911-7 (1959).
27. Morrison, W. R. & Smith, L. M. Preparation of Fatty Acid Methyl Esters and Dimethylacetals from Lipids with Boron Fluoride—Methanol. J Lipid Res 5, 600-8 (1964).
28. Salem, N., Jr., Reyzer, M. & Karanian, J. Losses of arachidonic acid in rat liver after alcohol inhalation. Lipids 31 Suppl, S153-6 (1996).
29. Masood, A., Stark, K. D. & Salem, N., Jr. A simplified and efficient method for the analysis of fatty acid methyl esters suitable for large clinical studies. J Lipid Res 46, 2299-305 (2005).
30. Grounds, M. D. et al. Silencing TNFalpha activity by using Remicade or Enbrel blocks inflammation in whole muscle grafts: an in vivo bioassay to assess the efficacy of anti-cytokine drugs in mice. Cell Tissue Res 320, 509-15 (2005).

1-27. (canceled)

28. A method for treating or preventing ocular neovascularization in a subject having or at risk for ocular neovascularization comprising administering to the subject an effective amount of omega-3 polyunsaturated fatty acid to thereby treat or prevent the ocular neovascularization.

29. The method of claim 28, wherein the ocular neovascularization is associated with the condition selected from the group consisting of retinopathy of prematurity, retina vein occlusion, sickle cell retinopathy, choroidal neovascularization, radiation retinopathy, microangiopathy, retinal hyperoxia, diabetic retinopathy, ablation induced neovascularisation, and age related macular degeneration.

30. The method of claim 28, wherein the ocular neovascularization results from retinopathy of prematurity, and the subject is a premature infant.

31. The method of claim 30, wherein the premature infant is exposed to high levels of oxygen.

32. The method of claim 28, wherein the omega-3 polyunsaturated fatty acid is administered in a high-dose.

33. A method of preventing vision loss arising from retinopathy of prematurity in a newborn comprising administering to the newborn afflicted with or at risk for retinopathy of prematurity, an effective amount of omega-3 polyunsaturated fatty acid to thereby prevent vision loss arising from retinopathy of prematurity.

34. A method for treating or preventing retinopathy of prematurity in a premature newborn comprising selecting a premature newborn and administering to the premature newborn an effective amount of omega-3 polyunsaturated fatty acid to thereby treat or prevent the retinopathy of prematurity.

35. The method of claim 34, wherein the newborn is a premature infant.

36. The method of claim 34, wherein the newborn is exposed to high levels of oxygen.

37. The method of claim 34, wherein the omega-3 polyunsaturated fatty acid is administered to the newborn periodically until the eye is fully vascularized.

38. The method of claim 34, wherein the omega-3 polyunsaturated fatty acid is administered in a regimen over a period of time between birth and the age of one year.

39. The method of claim 34, wherein the omega-3 polyunsaturated fatty acid is docosahexaenoic acid or eicosapentaenoic acid, or any combination thereof.

40. The method of claim 34, wherein the omega-3 polyunsaturated fatty acid is administered in a pharmaceutically acceptable carrier.

41. The method of claim 34, wherein the omega-3 polyunsaturated fatty acid is administered in a high-dose.

42. The method of claim 34, wherein the omega-3 polyunsaturated fatty acid is in a fatty emulsion.

43. The method of claim 42, wherein the emulsion is free of plant derived omega-6 fatty acid.

44. The method of claim 43, wherein the emulsion comprises fish oil.

45. The method of claim 34, wherein the omega-3 polyunsaturated fatty acid is administered to the newborn periodically until the eye is fully vascularized.

46. The method of claim 34, wherein the omega-3 polyunsaturated fatty acid is administered orally.

47. The method of claim 34, wherein the omega-3 polyunsaturated fatty acid is administered intravenously.
